(19) United States
(12) Patent Application Publication
(10) Pub. No.: US 2024/0240259 A1
(43) Pub. Date: Jul. 18, 2024

(54) METHODS AND COMPOUNDS FOR NEOANTIGEN VACCINES

(71) Applicant: THE TRANSLATIONAL GENOMICS RESEARCH INSTITUTE, Phoenix, AZ (US)

(72) Inventors: Sunil Sharma, Phoenix, AZ (US); Raffaella Soldi, Phoenix, AZ (US); John Altin, Flagstaff, AZ (US); Jeffrey Trent, Phoenix, AZ (US)

(21) Appl. No.: 18/551,356

(22) PCT Filed: Mar. 26, 2022
(86) PCT No.: PCT/US2022/022074

§ 371 Date: Sep. 19, 2023

Related U.S. Application Data
(60) Provisional application No. 63/166,697, filed on Mar. 26, 2021.

(51) Int. Cl. C12Q 1/6886; A61K 39/00; C12N 15/10

(52) U.S. Cl. CPC C12Q 1/6886; A61K 39/0011; C12N 15/1037; A61K 2039/53; A61K 2039/585; C12Q 2600/156

(57) ABSTRACT

The present disclosure includes compounds and methods for treating a subject having a disease such as cancer. A treatment method includes administering to the patient a therapeutically effective amount of one or more peptides corresponding to a tumor neoantigen or administering to the patient a therapeutically effective amount of one or more oligonucleotides each having a nucleic acid sequence that encodes a peptide corresponding to a tumor neoantigen. The tumor neoantigens may be identified from patient-specific tumor mutations in the patient's tumor cells.

### CROSS REFERENCE TO RELATED APPLICATIONS

This application claims the benefit of U.S. Provisional Patent Application No. 63/166,697, filed on Mar. 26, 2021, the contents of which are incorporated herein by reference in its entirety.

### REFERENCE TO SEQUENCE LISTING SUBMITTED ELECTRONICALLY

The official copy of the sequence listing is submitted electronically via EFS-Web as an ASCII-formatted sequence listing with a file named “91482-255WO-PCT_Sequence_Listing.txt” created on Mar. 25, 2022, and having a size of 7,379 bytes, is filed concurrently with the specification. The sequence listing contained in this ASCII-formatted document is part of the specification and is herein incorporated by reference in its entirety.

### TECHNICAL FIELD

The present invention relates to novel proteins and methods for their preparation and use as therapeutic or prophylactic agents, for example for treatment of cancer.

### BACKGROUND

Cancer is a group of diseases involving abnormal cell growth with a potential to spread to various parts of the body. Hundreds of types of cancers affect humans, and millions of people have been diagnosed and millions more are being diagnosed every year. The most common types of cancers include lung cancer, breast cancers, prostate cancers, colorectal cancers, among others. Treatment for cancers includes surgery, radiation therapy, chemotherapy, immunotherapy, hormone therapy, and stem cell replacement. Treatment options can be invasive and have a variety of undesirable side effects.

Accordingly, while the scientific community has made progress in this field, there remains a need in the art for improved compounds and methods for prevention and treatment of cancer.

### SUMMARY

A need exists for a therapeutic strategy for treatment of disease, including cancer. In order to actively drive an antitumor immune response, therapeutic cancer vaccines have been developed. Unlike prophylactic vaccines that are used preventatively to treat infectious diseases, therapeutic vaccines are designed to treat established cancer by stimulating an immune response against a specific tumor-associated antigen. The advantage of active immunotherapies is that they have the potential to provide long-lasting anticancer activity by engaging both the innate and adaptive arms of the immune response. Neoantigens are Major Histocompatibility Complex (MHC)-presented peptides of novel sequence that are formed by somatic mutation and represent an especially promising immunotherapeutic target, since they appear exclusively on the tumor and so cause minimal off-target effects.

The present invention provides a method for treating cancer in a patient. A treatment method may include administering to the patient a therapeutically effective amount of one or more peptides corresponding to tumor neoantigens or administering to the patient a therapeutically effective amount of one or more oligonucleotide each having a nucleic acid sequence that encodes a peptide corresponding to a tumor neoantigen. The neoantigens may be identified from patient-specific tumor mutations in the patient's tumor cells.

According to various embodiments, the method may comprise: (a) obtaining tumor cells from a tumor resected from the patient; (b) detecting a plurality of patient-specific tumor mutations in the tumor cells with a genomic analysis of tumor DNA and/or RNA and normal DNA and/or RNA from the patient; (c) identifying neoantigens resulting from somatic mutations that demonstrate specific binding with human leukocyte antigen (HLA) proteins or fragments thereof corresponding to a genotype of the patient; and (d) administering to the patient a therapeutically effective amount of the peptides corresponding to the tumor neoantigens (“neoantigen peptides”). An adjuvant may be administered to the patient concurrently with the neoantigens peptides.

According to other embodiments, the method may comprise: (a) obtaining tumor cells from a tumor resected from the patient; (b) detecting a plurality of patient-specific tumor mutations in the tumor cells with a genomic analysis of tumor DNA and/or RNA and normal DNA and/or RNA from the patient; (c) identifying a neoantigen resulting from somatic mutations that demonstrate specific binding with human leukocyte antigen (HLA) proteins or fragments thereof corresponding to a genotype of the patient; (d) designing a peptide based on the neoantigen; (e) generating a nucleic acid sequence that encodes the peptide; and (f) administering to the patient a therapeutically effective amount of an oligonucleotide having the nucleic acid sequence that encodes the peptide.

In certain aspects, detecting a plurality of patient-specific tumor mutations may comprise genomic profiling with next generation sequencing of a targeted gene panel. In one aspect, the genomic profiling comprises whole genome profiling, whole exome profiling, and/or transcriptome profiling.

In other aspects, the genomic analysis may comprise identifying a plurality of patient-specific tumor mutations in expressed genes by nucleic acid sequencing of tumor and normal samples from the patient and the mutations are present in the genome of cancer cells of the patient but not in normal cells from the subject.

In yet other aspects, the plurality of patient-specific tumor mutations may comprise a point mutation, splice-site mutation, frameshift mutation, read-through mutation, gene-fusion mutation, insertion, deletion, or a combination thereof; and the plurality of patient-specific tumor mutations encodes at least one mutant polypeptide having a tumor-specific neoepitope which binds to an HLA protein or fragment thereof with a greater affinity than a wild-type polypeptide.

In some aspects, the method may further comprise identifying the MHC class 1 and 2 genotypes of the patient. In one aspect, identifying the MHC class 1 and 2 genotypes of the patient comprises analysis of whole exome sequencing (WES) and/or RNA sequencing from tumor and/or normal tissue.

In certain aspects, identifying the neoantigens may comprise: (i) providing a library of peptide constructs, wherein each peptide construct of the library comprises a peptide portion and an identifying nucleic acid portion that identifies the peptide portion, and the peptide portion of at least one of the peptide constructs is capable of specific binding to the HLA proteins or fragments thereof; (ii) contacting the HLA proteins or fragments thereof with the library of peptide constructs; (iii) separating the at least one peptide construct comprising a peptide portion capable of specific binding to the HLA proteins or fragments thereof from peptide constructs comprising a peptide portion not capable of specific binding to the HLA proteins or fragments thereof; (iv) sequencing all or a portion of the identifying nucleic acid portion of the at least one peptide construct capable of specific binding to the HLA proteins or fragments thereof.

In some aspects, the library of peptide constructs may comprise variant peptides designed from an analysis of the plurality of patient-specific tumor mutations predicting the impact of each mutation on a corresponding protein and excluding silent mutations and mutations in noncoding regions. In one aspect, the variant peptides comprise mutations predicted to impact the structure of the corresponding protein.

In other aspects, identifying the neoantigens may comprise: (i) generating a genetically encoded combinatorial library of polypeptides with phage display, ribosomal display, mRNA display, biscistronic DNA display, P2A DNA display, CIS display, yeast display, or bacterial display, wherein the combinatorial library comprises polypeptides linked to corresponding nucleic acid molecules encoding the polypeptides; (ii) contacting the combinatorial library with the HLA proteins or fragments thereof, (iii) separating HLA proteins or fragments thereof demonstrating specific binding with the combinatorial library; and (iv) sequencing all or a portion of the nucleic acid molecules of the combinatorial library bound to the HLA proteins or fragments thereof to identify the neoantigens.

In one aspect, the combinatorial library of polypeptides may comprise variant peptides designed from an analysis of the plurality of patient-specific tumor mutations predicting the impact of each mutation on a corresponding protein and excluding silent mutations and mutations in noncoding regions.

In certain aspects, specific binding between neoantigens and HLA proteins or fragments thereof may be determined by: (i) culturing a cell transformed with at least one nucleic molecule comprising a nucleotide sequence encoding: an MHC class II component comprising at least a portion of an MHC class II a chain and at least a portion of an MHC class II β chain, such that the MHC class II a chain and MHC class II β chain form a peptide binding groove; and a spaceholder molecule and a first processable linker, wherein the spaceholder molecule is linked to the MHC class II component by the processable linker and the spaceholder molecule binds within the peptide binding groove thereby hindering the binding of any other peptide within the peptide binding groove; the step of culturing being conducted to produce the MHC class II component; (ii) recovering the MHC class II component; (iii) processing the processable linker, thereby releasing the spaceholder molecule from the peptide binding groove; (iv) incubating the MHC class II component in the presence of a neoantigen, wherein the incubation facilitates the binding of the neoantigen to the peptide binding groove; (v) recovering the MHC class II component that has bound the neoantigen.

In one aspect, the spaceholder molecule may have the consensus sequence AAXAAAAAAAXAA (SEQ ID NO: 30). In another aspect, the spaceholder molecule is selected from the group consisting of PVSKMRMATPLLMQA (SEQ ID NO: 25); AAMAAAAAAAMAA (SEQ ID NO: 26); AAMAAAAAAAAAA (SEQ ID NO: 27); AAFAAAAAAAAAA (SEQ ID NO: 28); and ASMSAASAASMAA (SEQ ID NO: 29).

In some aspects, the processable linker is linked to the MHC class II a chain of the MHC class II component. In other aspects, recovering the MHC class II component with bound neoantigen comprises affinity chromatography with an antibody recognizing the MHC class II component.

In certain aspects, specific binding between neoantigens and HLA proteins or fragments thereof is determined by phage display, the HLA proteins or fragments thereof are expressed on the surface of a phage, and the neoantigens are incubated with the phage to assay specific binding.

In one aspect, the method further comprises an in silico analysis to determine specific binding between neoantigens and MHC class I proteins or fragments thereof, wherein the in silico analysis comprises applying a computational algorithm to predict relative binding to MHC I proteins based on the peptide sequences of the neoantigens.

In other aspects, the present invention relates to a method of treating cancer in a patient, the method comprising: obtaining tumor cells from a tumor resected from the patient; detecting a plurality of patient-specific tumor mutations in the tumor cells with a genomic analysis of tumor DNA and/or RNA and normal DNA and/or RNA from the patient; identifying neoantigens resulting from somatic mutations that demonstrate specific binding with human leukocyte antigen (HLA) proteins or fragments thereof corresponding to a genotype of the patient; generating a messenger ribonucleic acid (mRNA) polynucleotide comprising an open reading frame encoding one or more peptides based on the neoantigens; and administering to the patient a therapeutically effective amount of the mRNA polynucleotide.

In some aspects, the mRNA polynucleotide comprises at least one chemical modification. In one aspect, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-i-methyl-pseudouridine, 4-thio-pseudouridine, 5-aza-uridine, dihydropseudouridine, 5-methoxyuridine and 2′-O-methyl uridine. In another aspect, the chemical modification is in the carbon 5-position of the uracil. In another aspect, the chemical modification is a N1-methylpseudouridine or N1-ethylpseudouridine.

In certain aspects, at least 80% of the uracils in the open reading frame have a chemical modification. In other aspects, the mRNA polynucleotide further encodes a 5′ terminal cap. In one aspect, the 5′ terminal cap is 7mG(5′)ppp(5′)NlmpNp.

In other aspects, the disclosed methods further comprise administering an effective amount of a therapeutic population of tumor infiltrating lymphocytes (TILs). In some aspects, the therapeutic population of TILs is administered simultaneously or sequentially with the peptides or mRNA polynucleotide. the therapeutic population of TILs has been activated and/or educated by at least one neoantigen presented by the peptides or encoded by the mRNA polynucleotide.

In some aspects, administering the peptides or mRNA polynucleotide with the therapeutic population of TILs enhances the immunogenic response and/or anti-tumor activity in the patient. In one aspect, the increase in the immunogenic response and/or anti-tumor activity in the patient is synergistic.

The foregoing features and elements may be combined in various combinations without exclusivity, unless expressly indicated otherwise. These features and elements as well as the operation thereof will become more apparent in light of the following description. It should be understood, however, the following description is intended to be exemplary in nature and non-limiting.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the figures, wherein like numerals may denote like elements.

FIG. 1 illustrates a method that uses genomic tools to produce a personalized neoantigen vaccine;

FIG. 2 illustrates an overview of an approach for development of a neoantigen peptide-based vaccine and a method of treating a patient using the neoantigen peptide-based vaccine;

FIG. 3 illustrates a step-wise work flow for efficient synthesis and analysis of large libraries of DNA-barcoded peptides; the peptide-DNA library depicted shows a puromycin (“Puro”) adaptor that facilitates linking of the DNA to the encoded peptide;

FIG. 4 illustrates analysis of tumor exomes to identify candidate neoantigens, characterization of the candidate neoantigens with the MHC-peptide-DNA conjugate assay, and evaluation of T cell response to the neoantigens that bound to HLA proteins in the MHC-peptide-DNA conjugate assay;

FIG. 5 illustrates a DNA conjugate assay library design for the analysis of mutations identified from an analysis of tumor exomes;

FIG. 6 illustrates an embodiment of the MHC-peptide-DNA conjugate assay used to identify neoantigens that bind to MHC Class II molecules; in the control group (“uncleaved”), the CLIP peptide is not removed from the HLA construct and candidate neoantigens bind non-specifically while in the test group (“cleaved”) removal of the CLIP peptide opens the recognition site of the HLA construct allowing for specific binding of candidate neoantigens;

FIG. 7 illustrates the identification of enriched peptides (i.e., neoantigens) from 2 human patients (i.e., TG0006 and TG00013); the numbers of mutations identified in each patient with exome analysis are shown under each patient ID; the human leukocyte antigen serotypes of the HLA complexes used in the binding assays are identified in the upper left-hand corner of each graph. Enriched peptides are shown as larger data points FIGS. 8A and 8B illustrate additional data sets identifying enriched peptides (i.e., neoantigens) from human patient TG00013; enriched peptides appear as data points in the upper portion of each graph corresponding to those peptides with a log average % read with cleaved HLA complexes closer to 0 (i.e., closer to 100% binding with cleaved HLA complexes);

FIG. 9 illustrates the amino acid sequences of the enriched peptides, the corresponding mutation for each peptide, and the human leukocyte antigen serotype of the HLA complex with which the peptide specifically binds for human patient TG00013; and

FIG. 10 illustrates a comparison of the MHC-peptide-DNA conjugate assay versus in silico prediction to identify neoantigens; the IEDB consensus sequences used for the in silico analysis are obtained from the Immune Epitope Database and Analysis Resource (IEDB), a public resource maintained by the National Institute of Allergy and Infectious Diseases (NIAID).

FIG. 11 illustrates information on several human patients who were evaluated by collecting tumor infiltrating lymphocytes (TILs) and generating peptides to activate and educate these TILs to enhance recognition of neoantigens and anti-tumor activity.

FIG. 12 illustrates a non-limiting example of peptide vaccine manufacturing with the disclosed methods.

FIG. 13 illustrates infiltration of tumor cell organoids by peripheral blood mononuclear cells (PBMC) derived from the patient with the tumor. The PBMCs were administered alone or with DMSO (negative controls), in combination with IL-2, or in combination with IL-2 and a peptide identified as a neoantigen. The images were captured 24 hours after the organoids were subjected to the various treatment. As shown in the bar graphs, after only 24 hours the PBMCs in combination the IL-2 and the peptide demonstrated significant infiltration of the organoid.

FIG. 14 illustrates a study to test the safety and efficacy of the disclosed peptide vaccines. ORR: objective response rate; DOR: duration of any observed response; PFS: progression-free survival; OS: overall survival.

### DETAILED DESCRIPTION

It is to be understood that unless specifically stated otherwise, references to “a,” “an,” and/or “the” may include one or more than one and that reference to an item in the singular may also include the item in the plural. Reference to an element by the indefinite article “a,” “an” and/or “the” does not exclude the possibility that more than one of the elements are present, unless the context clearly requires that there is one and only one of the elements. As used herein, the term “comprise,” and conjugations or any other variation thereof, are used in its non-limiting sense to mean that items following the word are included, but items not specifically mentioned are not excluded.

A “neoepitope” is understood in the art to refer to an epitope that emerges or develops in a subject after exposure to or occurrence of a particular event (e.g., development or progression of a particular disease, disorder or condition, e.g., infection, cancer, stage of cancer, etc.). As used herein, a neoepitope is one whose presence and/or level is correlated with exposure to or occurrence of the event. In some embodiments, a neoepitope is one that triggers an immune response against cells that express it (e.g., at a relevant level). In some embodiments, a neoepitope is one that triggers an immune response that kills or otherwise destroys cells that express it (e.g., at a relevant level). In some embodiments, a relevant event that triggers a neoepitope is or comprises somatic mutation in a cell. In some embodiments, a neoepitope is not expressed in non-cancer cells to a level and/or in a manner that triggers and/or supports an immune response (e.g., an immune response sufficient to target cancer cells expressing the neoepitope). In some embodiments, a neoepitope is a neoantigen.

The terms “peptide,” “polypeptide,” and “protein” are used interchangeably herein to refer to a polymeric form of amino acids of any length, chemically or biochemically modified or derivatized amino acids, and polypeptides having modified peptide backbones.

The term “peptide construct” as used herein, refers to a peptide of any length attached to an identifying oligonucleotide. The attachment may be via an intervening linker and the attachment may be covalent or non-covalent. The identifying oligonucleotide may be the message that was translated to form the peptide portion of the construct, or it may be any other sequence that is known and can be used to identify the attached peptide by sequencing. ‘Peptide construct sets’ refer to a pool of peptide constructs generated from a custom-designed set of oligonucleotides. The sets may contain as few as one copy per species of peptide construct but typically contain many copies of each peptide construct.

The term “amino acid” refers to naturally occurring and synthetic amino acids, as well as amino acid analogs and amino acid mimetics that function in a manner similar to naturally occurring amino acids. Naturally occurring amino acids are those encoded by the genetic code, as well as those amino acids that are later modified. Unnatural amino acids are not encoded by the genetic code and can, but do not necessarily have the same basic structure as a naturally occurring amino acid. “Amino acid analogs” refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to by either the three letter symbols or by the one-letter symbols recommended by the IUPAC, the IUAPC letter code are as follows: G=Glycine; A=Alanine; L=Leucine; M=Methionine; F=Phenylalanine; W=Tryptophan; K=Lysine; Q=Glutamine; E=Glutamic Acid; S=Serine; P=Proline; V=Valine; I=Isoleucine; C=Cysteine; Y=Tyrosine; H=Histidine; R=Arginine; N=Asparagine; D=Aspartic Acid; T=Threonine.

The terms “homologous” and “similar” refer to the relationship between proteins that possess a “common evolutionary origin,” including proteins from superfamilies (e.g., the immunoglobulin superfamily) and homologous proteins from different species. Such proteins (and their encoding genes) have sequence homology, as reflected by their sequence similarity, whether in terms of percent similarity or the presence of specific residues or motifs as conserved positions. In a specific embodiment, two peptide sequences are “substantially homologous or similar” when at least about 80%, or at least about 90%, or at least about 95) of the amino acids match over the defined lengths of the amino acid sequences.

The term “variants” applies to both amino acid and nucleic acid sequences. Because of the degeneracy of the genetic code, a large number of functionally identical nucleic acids encode any given protein. For instance, the codons GCA, GCC, GCG and GCU all encode the amino acid alanine. Variants may include individual substitutions, deletions or additions to a nucleic acid, peptide, polypeptide, or protein sequence which alters, adds or deletes a single amino acid or a small percentage of amino acids in the encoded sequence. “Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acids other than those indicated as conserved may differ in a protein so that the percent protein or amino acid sequence similarity between any two proteins of similar function may vary and may be, for example, from 70% to 99% as determined according to an alignment scheme. A “variant” also includes a polypeptide which has at least 60% amino acid identity as determined by BLAST or FASTA algorithms, preferably at least 75% most preferably at least 85%, and even more preferably at least 90%, and still more preferably at least 95%, and which has the same or substantially similar properties or functions as the native or parent protein to which it is compared. A particular variant is a “gain-of-function” variant, meaning a polypeptide variant in which the change of at least one given amino acid residue in a protein or enzyme improves a specific function of the polypeptide, including, but not limited to protein activity. The change in amino acid residue can be replacement of an amino acid with one having similar properties.

As used herein, the term “binding” refers to an attractive interaction between two molecules which results in a stable association in which the molecules are in close proximity to each other. Molecular binding can be classified into the following types: non-covalent, reversible covalent and irreversible covalent. Molecules that can participate in molecular binding include proteins, nucleic acids, carbohydrates, lipids, and small organic molecules such as pharmaceutical compounds. For example, proteins that form stable complexes with other molecules are often referred to as receptors while their binding partners are called ligands. Nucleic acids can also form stable complex with themselves or others, for example, DNA-protein complex, DNA-DNA complex, DNA-RNA complex, protein-protein complex.

As used herein, the term “specific binding” refers to the specificity of a binder, e.g., a protein or an antibody, such that it preferentially binds to a target, such as a polypeptide antigen, a receptor, or an antibody. When referring to a binding partner, e.g., protein, nucleic acid, antibody or other affinity capture agent, etc., “specific binding” can include a binding reaction of two or more binding partners with high affinity and/or complementarity to ensure selective hybridization under designated assay conditions. Typically, specific binding will be at least three times the standard deviation of the background signal. Thus, under designated conditions the binding partner binds to its particular target molecule and does not bind in a significant amount to other molecules present in the sample. Recognition by a binder or an antibody of a particular target in the presence of other potential interfering substances is one characteristic of such binding. Preferably, binders, antibodies or antibody fragments, peptides, or fusion peptides that are specific for or bind specifically to a target bind to the target with higher affinity than binding to other non-target substances. Also preferably, binders, antibodies or antibody fragments, peptides, or fusion peptides that are specific for or bind specifically to a target avoid binding to a significant percentage of non-target substances, e.g., non-target substances present in a testing sample. The binding affinity of an antibody to a target antigen, antigenic fragment, peptide, or fusion peptide, comprising the cognate epitope can be readily determined using any of a number of methods available in the art including, but not limited to, enzyme linked immunosorbent assay (ELISA). In some embodiments, binders, antibodies or antibody fragments, peptides, or fusion peptides of the present disclosure avoid binding greater than about 90% of non-target substances, although higher percentages are clearly contemplated and preferred. For example, binders, antibodies or antibody fragments, or peptides, of the present disclosure avoid binding about 91%, about 92%, about 93%, about 94%, about 95%, about 96%, about 97%, about 98%, about 99%, and about 99% or more of non-target substances. In other embodiments, binders, antibodies or antibody fragments, or peptides of the present disclosure avoid binding greater than about 10%, 20%, 30%, 40%, 50%, 60%, or 70%, or greater than about 75%, or greater than about 80%, or greater than about 85% of non-target substances.

The terms “target,” “target molecule,” and “target agent” are used interchangeably herein and refer to a protein, toxin, enzyme, pathogen, cell or biomarker that is incubated with a library to identify peptides demonstrating specific binding to the target. A target or a marker may be any molecular structure produced by a cell, expressed inside the cell, accessible on the cell surface, or secreted by the cell. A marker may be any protein, carbohydrate, fat, nucleic acid, catalytic site, or any target of these such as an enzyme, glycoprotein, cell membrane, virus, cell, organ, organelle, or any uni- or multimolecular structure or any other such structure now known or yet to be disclosed whether alone or in combination. A target may also be called a marker and the terms are used interchangeably. A target may be represented by the sequence of amino acids, or sequence of one or more strands of a nucleic acid from which it may be derived. For example, a target may be represented by a protein sequence. Alternatively, a target may be represented by a nucleic acid sequence, the protein or peptide or the fragments thereof encoded by the nucleic acid sequence.

Examples of such nucleic acids include both single stranded and double stranded nucleic acid sequences including miRNA, tRNA, siRNA, mRNA, cDNA, or genomic DNA sequences including complimentary sequences. The concept of a marker is not limited to the products of the exact nucleic acid sequence or protein sequence by which it may be represented. Rather, a marker encompasses all molecules that may be detected by a method of assessing the expression of the marker. Examples of molecules encompassed by a marker include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The term “target” further encompasses the products (i.e., proteins) of the gene or a gene allele thereof, whose expression or activity is directly or indirectly associated with a particular phenotype or cellular condition, or physiological characteristic.

The terms “capture agent” and “capture group” as used herein refer to any moiety that allows capture of a target molecule or a peptide construct via binding to or linkage with an affinity group or domain on the target molecule or an affinity tag of the peptide construct. The binding between the capture agent and its affinity tag may be a covalent bond and/or a non-covalent bond. A capture agent includes, e.g., a member of a binding pair that selectively binds to an affinity tag on a fusion peptide, a chemical linkage that is added by recombinant technology or other mechanisms, co-factors for enzymes and the like. Capture agents can be associated with a peptide construct using conventional techniques including hybridization, cross-linking (e.g., covalent immobilization using a furocoumarin such as psoralen), ligation, attachment via chemically-reactive groups, introduction through post-translational modification and the like. “Sequence determination,” “sequencing,” and the like include determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput sequencing” or “next generation sequencing” includes sequence determination using methods that determine many (typically thousands to billions) of nucleic acid sequences in an intrinsically parallel manner, i.e., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, CT); sequencing by ligation (for example, as commercialized in the SOLiD™ technology, Life Technologies, Inc., Carlsbad, CA); sequencing by synthesis using modified nucleotides (such as commercialized in TruSeq™ and HiSeq™ technology by Illumina, Inc., San Diego, CA; HeliScope™ by Helicos Biosciences Corporation, Cambridge, MA; and PacBio RS by Pacific Biosciences of California, Inc., Menlo Park, CA), sequencing by ion detection technologies (such as Ion Torrent™ technology, Life Technologies, Carlsbad, CA); sequencing of DNA nanoballs (Complete Genomics, Inc., Mountain View, CA); nanopore-based sequencing technologies (for example, as developed by Oxford Nanopore Technologies, LTD, Oxford, UK), and like highly parallelized sequencing methods.

The term “exome” is used in accordance with its art-understood meaning referring to the set of exon sequences that are found in a particular genome.

As used herein, the term “mutation” refers to permanent change in the DNA sequence that makes up a gene. In some embodiments, mutations range in size from a single DNA building block (DNA base) to a large segment of a chromosome. In some embodiments, mutations can include missense mutations, frameshift mutations, duplications, insertions, nonsense mutation, deletions and repeat expansions. In some embodiments, a missense mutation is a change in one DNA base pair that results in the substitution of one amino acid for another in the protein made by a gene. In some embodiments, a nonsense mutation is also a change in one DNA base pair. Instead of substituting one amino acid for another, however, the altered DNA sequence prematurely signals the cell to stop building a protein. In some embodiments, an insertion changes the number of DNA bases in a gene by adding a piece of DNA. In some embodiments, a deletion changes the number of DNA bases by removing a piece of DNA. In some embodiments, small deletions may remove one or a few base pairs within a gene, while larger deletions can remove an entire gene or several neighboring genes. In some embodiments, a duplication consists of a piece of DNA that is abnormally copied one or more times. In some embodiments, frameshift mutations occur when the addition or loss of DNA bases changes a gene's reading frame. A reading frame consists of groups of 3 bases that each code for one amino acid. In some embodiments, a frameshift mutation shifts the grouping of these bases and changes the code for amino acids. In some embodiments, insertions, deletions, and duplications can all be frameshift mutations. In some embodiments, a repeat expansion is another type of mutation. In some embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In some embodiments, a repeat expansion is a mutation that increases the number of times that the short DNA sequence is repeated.

“Small molecule,” as used herein, means a molecule less than 5 kilodaltons, more typically, less than 1 kilodalton. As used herein, “small molecule” includes peptides.

“Affinity tag” is given its ordinary meaning in the art. An affinity tag is any biological or chemical material that can readily be attached to a target biological or chemical material. Affinity tags may be attached to a target biological or chemical molecule by any suitable method. For example, in some embodiments, the affinity tag may be attached to the target molecule using genetic methods. For example, the nucleic acid sequence coding the affinity tag may be inserted near a sequence that encodes a biological molecule; the sequence may be positioned anywhere within the nucleic acid that enables the affinity tag to be expressed with the biological molecule, for example, within, adjacent to, or nearby. In other embodiments, the affinity tag may also be attached to the target biological or chemical molecule after the molecule has been produced (e.g., expressed or synthesized). As one example, an affinity tag such as biotin may be chemically coupled, for instance covalently, to a target protein or peptide to facilitate the binding of the target to streptavidin.

Affinity tags include, for example, metal binding tags such as histidine tags, GST (in glutathione/GST binding), streptavidin (in biotin/streptavidin binding). Other affinity tags include Myc or Max in a Myc/Max pair, or polyamino acids, such as polyhistidines. At various locations herein, specific affinity tags are described in connection with binding interactions. The molecule that the affinity tag interacts with (i.e., binds to), which may be a known biological or chemical binding partner, is the “recognition entity.” It is to be understood that the invention involves, in any embodiment employing an affinity tag, a series of individual embodiments each involving selection of any of the affinity tags described herein.

A “recognition entity” may be any chemical or biological material that is able to bind to an affinity tag. A recognition entity may be, for example, a small molecule such as maltose (which binds to MBP, or maltose binding protein), glutathione, NTA/Ni2+, biotin (which may bind to streptavidin), or an antibody. An affinity tag/recognition entity interaction may facilitate attachment of the target molecule, for example, to another biological or chemical material, or to a substrate (e.g., a nitrocellulose membrane or other immobilized substrate). Examples of affinity tag/recognition entity interactions include polyhistidine/NTA/Ni2+, glutathione S-transferase/glutathione, maltose binding protein/maltose, streptavidin/biotin, biotin/streptavidin, antigen (or antigen fragment)/antibody (or antibody fragment), and the like.

The term “ribosomal display” refers to a reaction system able to yield a ternary complex of an mRNA, ribosome and corresponding protein of interest. Ribosomal display can be used for screening cell surface receptors, antibodies, and fragments thereof for target antigen or ligand binding. The steps of producing the reaction system can include: 1) generating a DNA library and transcribing the library into an RNA library, 2) purifying the RNA and in vitro translation in a cell-free protein synthesis system, 3) allowing the ribosome complexes of the translation reaction to bind to a target antigen or ligand, 4) selecting bound ribosome complexes; and 5) isolating RNA from the complexes and reverse transcribing the transcripts to cDNA, wherein the cDNA can be amplified, sequenced and/or further modified.

In some embodiments, the pharmaceutical composition includes one or more peptides or oligonucleotides, as described herein, together with a pharmaceutically acceptable carrier, diluent or excipient. In the preparation of the pharmaceutical compositions comprising the peptides or oligonucleotides described in the teachings herein, a variety of vehicles, vectors, excipients, and routes of administration may be used. The pharmaceutical compositions will generally comprise a pharmaceutically acceptable carrier and a pharmacologically (or therapeutically) effective amount of the peptides or oligonucleotides. In various embodiments, the pharmaceutical compositions comprising the one or more peptides may include an adjuvant or other agent for inducing or enhancing a patient's immune response. In other embodiments, the pharmaceutical compositions comprising the one or more oligonucleotides may be delivered without an adjuvant.

The pharmaceutical compositions described herein may be administered by any means that enables the active agent to reach the agent's site of action in the body of the subject. The dosage administered varies depending upon factors, such as: pharmacodynamic characteristics; mode and route of administration; age, health, and weight of the recipient subject; nature and extent of symptoms; concurrent treatments; and frequency of treatment.

As used herein, the terms “administration” and “administering” of an agent to a subject include any route of introducing or delivering the agent to a subject to perform its intended function. Administration can be carried out by any suitable route, including intravenously, intramuscularly, intraperitoneally, inhalationally, intranasally, or subcutaneously. Administration includes self-administration and the administration by another.

The term “effective amount” or “therapeutically effective amount” refers to that amount of an agent or combination of agents as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells. The specific dose will vary depending on the particular agents chosen, the dosing regimen to be followed, whether the agent is administered in combination with other agents, timing of administration, the tissue to which it is administered, and the physical delivery system in which the compound is carried.

The terms “treatment,” “treating,” “treat,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “patient” or “subject” refers to any organism to which a provided composition is or may be administered, e.g., for experimental, diagnostic, prophylactic, cosmetic, and/or therapeutic purposes. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a human.

In some embodiments, a patient is suffering from or susceptible to one or more disorders or conditions. In some embodiments, a patient displays one or more symptoms of a disease, disorder or condition. In some embodiments, a patient has been diagnosed with one or more diseases, disorders or conditions. In some embodiments, the disorder or condition is or includes cancer, or the presence of one or more tumors.

Detection of Tumor Mutations and/or Neoepitopes

Cancers may be screened to detect mutations and/or neoepitopes (e.g., to detect neoantigen identity, and/or neoepitope nature, level, and/or frequency) as described herein using any of a variety of known technologies. In some embodiments, particular mutations or neoepitopes, or expression thereof, is/are detected at the nucleic acid level (e.g., in DNA or RNA). One of skill in the art would recognize that mutations or neoepitopes, or expression thereof, can be detected in a sample comprising DNA or RNA from cancer cells. Further, one of skill in the art would understand that a sample comprising DNA or RNA from cancer cells can include but is not limited to circulating tumor DNA (ctDNA), cell free DNA (cfDNA), cells, tissues, or organs. In some embodiments, mutations or neoepitopes, or expression thereof, is detected at the protein level (e.g., in a sample comprising polypeptides from cancer cells, which sample may be or comprise polypeptide complexes or other higher order structures including but not limited to cells, tissues, or organs).

In some particular embodiments, detection involves nucleic acid sequencing. In some embodiments, detection involves whole exome sequencing. In some embodiments, detection involves an immunoassay. In some embodiments, detection involves use of a microarray. In some embodiments, detection involves massively parallel exome sequencing. In some embodiments, detection involves genome sequencing. In some embodiments, detection involves RNA sequencing. In some embodiments, detection involves standard DNA or RNA sequencing. In some embodiments, detection involves mass spectrometry.

In some embodiments, detection involves next generation sequencing (DNA and/or RNA). In some embodiments, detection involves genome sequencing, genome resequencing, targeted sequencing panels, transcriptome profiling (RNA-Seq), DNA-protein interactions (ChIP-sequencing), and/or epigenome characterization. In some embodiments, re-sequencing of a patient's genome may be utilized, for example to detect genomic variations.

In some embodiments, detection involves using a technique such as ELISA, western blotting, immunoassay, mass spectrometry, microarray analysis, etc.

In some embodiments, detection involves next generation sequencing (DNA and/or RNA). In some embodiments, detection involves next generation sequencing of targeted gene panels (e.g. the ASHION® tumor/normal exome-RNA test (GEMEXTRA®), MSK-IMPACT, or FOUNDATIONONE®). In some embodiments, detection involves genomic profiling.

In some embodiments, detection involves genomic profiling using the GEMEXTRA® test. The GEMEXTRA®test is a comprehensive exome and transcriptome profiling assay that both informs the care of cancer patients and enables future research into the disease. Greater than 19,000 genes are assayed via hybridization capture and clinical-depth sequencing for the identification of somatic point mutations, small and large insertions and deletions and structural rearrangements. Measures of microsatellite instability (MSI) and tumor mutational burden (TMB) are also taken to inform the application of immunoncology therapies. To ensure somatic origin of identified variants, both the germline and tumor exomes are sequenced and compared. The entire transcriptome is also sequenced, enabling the detection of gene fusion and alternative splicing events from the patient's RNA. GEMEXTRA® is applicable to both solid and hematological cancers.

In some embodiments, detection involves genomic profiling using Integrated Mutation Profiling of Actionable Cancer Targets (MSK-IMPACT) (see Cheng D T, Mitchell T N, Zehir A, et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn. 2015; 17(3):251-264; and Ross D S, Zehir A, Cheng D T, et al. Next-Generation Assessment of Human Epidermal Growth Factor Receptor 2 (ERBB2) Amplification Status: Clinical Validation in the Context of a Hybrid Capture-Based, Comprehensive Solid Tumor Genomic Profiling Assay. J Mol Diagn. 2017; 19(2):244-254). MSK-IMPACT is a comprehensive molecular profiling assay that involves hybridization capture and deep sequencing of all exons and selected introns of multiple oncogenes and tumor-suppressor genes, allowing for detection of point mutations, small and large insertions or deletions, and rearrangements. MSK-IMPACT also captures intergenic and intronic single-nucleotide polymorphisms (e.g., tiling probes), interspersed across a genome, aiding in accurate assessment of genome-wide copy number. In some embodiments, probes may target a megabase.

In some embodiments, detection involves genomic profiling using the FOUNDATIONONE® CDX^{1M} (“FlCDx”) assay. The FlCDx assay is a next generation sequencing based in vitro diagnostic device for detection of substitutions, insertion and deletion alterations (indels), and copy number alterations (CNAs) in 324 genes and select gene rearrangements, as well as genomic signatures including microsatellite instability (MSI) and tumor mutation burden (TMB) using DNA isolated from formalin-fixed paraffin embedded (FFPE) tumor tissue specimens. FlCDx is approved by the United States Food and Drug Administration (FDA) for several tumor indications, including NSCLC, melanoma, breast cancer, colorectal cancer, and ovarian cancer.

The FlCDx assay employs a single DNA extraction method from routine FFPE biopsy or surgical resection specimens, 50-1000 ng of which will undergo whole-genome shotgun library construction and hybridization-based capture of all coding exons from 309 cancer-related genes, one promoter region, one non-coding (ncRNA), and selected intronic regions from 34 commonly rearranged genes, 21 of which also include the coding exons. In total, the assay detects alterations in a total of 324 genes. Using the ILLUMINA® HiSeq 4000 platform, hybrid capture-selected libraries are sequenced to high uniform depth (targeting >500× median coverage with >99% of exons at coverage >100×). Sequence data is then processed using a customized analysis pipeline designed to detect all classes of genomic alterations, including base substitutions, indels, copy number alterations (amplifications and homozygous gene deletions), and selected genomic rearrangements (e.g., gene fusions). Additionally, genomic signatures including microsatellite instability (MSI) and tumor mutation burden (TMB) are reported.

In some embodiments, detection may involve sequencing of exon and/or intron sequences from at least 100, 150, 200, 250, 300, 350, 400, 450, 500, 550, 600, 650, 700, 750, 800, 850, 900, 950, 1000 or more genes (e.g., oncogenes and/or tumor-suppressor genes). For example, literature reports indicate that MSK-IMPACT has been used to achieve deep sequencing of all exons and selected introns of 468 oncogenes and tumor-suppressor genes. Alternatively or additionally, in some embodiments, detection may involve sequencing of intergenic and/or intronic single-nucleotide polymorphisms. For example, literature reports indicate that MSK-IMPACT has been used to achieve deep sequencing of >1000 intergenic and intronic single-nucleotide polymorphisms.

In some embodiments, detection involves sequencing of splice variants. Cancer cells are able to adapt and evolve by developing mechanisms to escape control by their microenvironment. The diversity and plasticity offered by alternative splicing, therefore, provide an opportunity for cancer cells to produce protein isoforms suitable for tumor growth and/or spreading (David C J and Manley J L. Genes Dev. 2010; 24(21):2343-2364). Genome-wide approaches has revealed that large-scale alternative splicing occurs during tumorigenesis (Venables J P et al. Nat Struct Mol Biol. 2009; 16(6):670-676.), and the genomic portraits of alternative splicing patterns have proven useful in the classification of tumors (Venables J P. Bioessays. 2006; 28(4):378-386; Skotheim R I et al., Int J Biochem Cell Biol. 2007; 39(7-8):1432-1449; Omenn G S et al., Dis Markers. 2010; 28(4):241-251).

Reports of aberrant splicing events and alterations in ratios of alternatively spliced transcripts in different cancers have been noted (Rajan P et al., Nat Rev Urol. 2009; 6(8):454-460). These events result in novel transcripts not observed in normal cell counterparts. It has been reported that nearly all areas of tumor biology are affected by alternative splicing, including metabolism, apoptosis, cell cycle control, invasion, metastasis and angiogenesis (Venables J P., Bioessays. 2006; 28(4):378-386; Ghigna C et al., Curr Genomics. 2008; 9(8):556-570).

One of the earliest examples of alternative splice variants with opposing apoptotic effects is Bcl-x. The Bcl-x pre-mRNA can be alternatively spliced to produce two splice variants, anti-apoptotic Bcl-xL (long form) and pro-apoptotic Bcl-xS (short form) (Boise L H et al. Cell. 1993; 74(4):597-608). High Bcl-xL/Bcl-xS ratios, promoting tumor cell survival, can be found in a number of cancer types, including human lymphoma, breast cancer, and human hepatocellular carcinoma (Minn A J et al., J Biol Chem. 1996; 271(11):6306-6312.44-46; Olopade O I et al., Cancer J Sci Am. 1997; 3(4):230-237; Takehara T et al., Hepatology. 2001; 34(1):55-61). Another example of an apoptosis-related gene that undergoes alternative splicing in cancer cells is the Fas receptor gene.

Expressed on the cell surface of many cell types, the Fas receptor is activated by the Fas ligand produced by cytotoxic T cells, which initiates a death-signaling cascade leading to apoptosis of cells expressing the Fas receptor (Bouillet P et al, Nat Rev Immunol. 2009; 9(7):514-519). There are at least 3 short mRNA variants of Fas missing the encoded transmembrane domain and the resulting translated protein variants are presumably secreted by cancer cells and act as decoy receptors for the Fas ligand, thus allowing cancer cells to escape from apoptosis (Cheng J et al., Science. 1994; 263(5154):1759-1762; Cascino I et al., Journal of immunology. 1995; 154(6):2706-2713).

Alternative splicing of the H-Ras oncogene occurs on a previously unknown spliced exon (named as IDX) caused by an intronic mutation in the H-Ras gene (Cohen J B et al., Cell. 1989; 58(3):461-472). This mutation of the IDX splice site results in an H-Ras mRNA variant, which is more resistant to the nonsense-mediated mRNA decay (NMD) process, and consequently overexpressed in cancers (Barbier J et al., Mol Cell Biol. 2007; 27(20):7315-7333). Alternative splicing also plays a role in promoting invasive and metastatic behavior in cancers. CD44 was among the first genes with splice variants specifically associated with metastasis, where variants containing exons 4-7 (v4-7) and 6-7 (v6-7) were shown to be expressed in a metastasizing pancreatic carcinoma cell line, but not in the corresponding parental tumor (Gunthert U et al., Cell. 1991; 65(1):13-24).

### Mutational Calling Software Tools

In certain aspects, a Pegasus human research pipeline at TGen. https://github.com/tgen/pegasusPipe is used. The sequences are aligned to the HG19 human genome or the HG38 human genome. In one aspect, somatic mutations are called using three separate mutational callers: Seurat, Mutect, and Strelka. All mutations that were seen in more than one of the callers were selected and used to generate peptides. The rationale behind this is that each caller has its own strengths and weaknesses. By pulling mutations that are called by multiple callers the risk of identifying false positives is reduced. The callers are available at: Mutect:https://software.broadinstitute.org/cancer/cga/mutect (Broad Institute Software); Seurat: https://github.com/tgen/seurat (TGen Caller); and Strelka: https://github.com/Illumina/strelka (GNU general public license). Best practices are followed for DNA and RNA workflows. Alternatively, CLIA certified somatic calls are used with no changes to generate peptides as candidate neoantigens for further analysis.

In some embodiments, single or multiple mutation callers may be utilized to call genomic variants. For samples consisting of sequencing tumor and normal tissue the callers that may be used are: Strelka, Strelka2, VarDict, VarScan2, qSNP, Shimmer, RADIA, SOAPsnv, SomaticSniper, FaSD-somatic, Samtools, JointSNVMix, Virmid, SNVSniffer, Seurat, CaVEMan, MuTect, MuTect2, LoFreq, EBCall, deepSNV, LoLoPicker, MuSE, MutationSeq, SomaticSeq, SnooPer, FreeBayes, HapMuC, SPLINTER, Pisces, DeepSNVMiner, smCounter, DeepVariant, Cake, Tnscope, DNAscope, NeoMutate, Maftools, scABA, Sanivar, Sarek, BATCAVE, SomaticNet, CoVaCS, RegTools, xAtlas, R2D2, SiNPle, Bambino, and exactSNP. For samples consisting of tumor only sequencing callers that may be utilized are: Platypus, LumosVar, LumosVar2, SNVMix2, SNVer, OutLyzer, ISOWN, SomVarIUS, SiNVICT, FreeBayes, SNPiR, eSNV-dectect, RNAIndel, VaDir, and Clairvoyante.

In certain embodiments, single or multiple mutation callers recognizing splice variants may be utilized to call genomic variants. For samples consisting of sequencing tumor and normal tissue the callers that may be used are: RegTools, ASGAL, MATS (rMATS), SUPPA (SUPPA2), Leafcutter, MAJIQ, JunctionSeq, findAS, Cufflinks/Cuffdiff, IsoformSwitchAnalyzeR, ALEXA-seq, MISO, SplicingCompass, Flux Capacitor, JuncBASE, SpliceR, FineSplice, ARH-seq, Spladder, DEXSeq, edgeR, Limma, DiffSplice, dSpliceType, SpliceDetector, HISAT2, STAR, Subread, Subjunct, PennDiff, DSGseq, AltAnalyze, Splicing Express, SpliceTrap, PSGInfer, FDM, IsoEM2, MADS+, Spanki, SpliceMap, CASPER, AVISPA, PASA, SpliceSeq, MATT, Aspli, IPSA, SANJUAN, VAST-Tools, SpliceV, Asprofile, DESeq2, Yanagi, ABLas, IRIS, and AS-Quant.

### HLA Typing

In some embodiments, whole exome sequencing (WES) and/or RNA sequencing from tumor and/or normal tissue may be used to identify the MHC class 1 and 2 genotypes for individual patients.

Single or multiple HLA callers may be utilized to identify the HLA types. For DNA sequencing the HLA callers that may be utilized are: BWAkit, PHLAT, OptiType, HLAMiner, HISAT, HISAT2, xHLA, HLA-Vbseq, GATK HLA Caller, PolySolver, HLAReporter, Athlates, HLAseq, HLAssign, SOAP-HLA, STC-SEq, HapLogic, Gyper, HLATyphon, HLA-LA, HLA-PRG, MHC-PRG, Prohlatype, GraphTyper, ALPHLARD, Omixon, SNP2HLA, HLAscan, NeoEpitopePred, and Assign. For RNA sequencing the HLA callers that may be utilized are: BWAkit, seq2HLA, PHLAT, HLAProfiler, OptiType, HLAMiner, HLAForest, arcasHLA, HISAT, HISAT2, HLAseq, HLAssign, STC-Seq, HLATyphon, ALPHLARD, Omixon, and HLAscan.

In some embodiments, whole exome sequencing data may be analyzed for HLA typing using Polysolver (polymorphic loci resolver) (Shukla et al., 2015). Polysolver is an algorithm for inferring alleles of the three major MHC class I (HLA-A, HLA-B, HLA-C) genes. For example, a patient's HLA class I alleles may be inferred from whole exome sequencing data using Polysolver. In other variations, in the absence of whole exome sequencing data, a patient's HLA class I alleles may be inferred from transcriptome sequencing data by an alternative HLA typing algorithm.

In one embodiment, HLA typing is conducted in silico using one or more techniques such as OptiType, run on a computing device. See Szolek, et al., OptiType: precision HLA typing from next-generation sequencing data, Bioinformatics. 2014 Dec. 1; 30(23), incorporated herein in its entirety for all purposes. A variety of other in silico techniques may also be used. See Major, et al., HLA typing from 1000 genomes whole genome and whole exome Ilumina data, PLoS One. 2013 Nov. 6; 8(1 l):e78410; Wittig, et al., Development of a high-resolution NGS-based HLA-typing and analysis pipeline, Nucl. Acids Res. (2015).

Generation of Peptides from Tumor Mutations

In some embodiments mutational data is acquired as described above in the section entitled “Detection of Tumor Mutations and/or Neoepitopes”. The mutational calls may be converted to mutant protein sequences using single or multiple tools including but not limited to: Varcode, customProDB, and pyGeno. The tools will generate the peptides sequences for downstream in-vitro and in-silico HLA binding assays. Additionally, in some embodiments, the peptides will undergo testing to validate that each peptide is unique to the human proteome. The peptides may be aligned to several different protein databases from NCBI including: Non-redundant protein sequences, Reference proteins, model organisms, UniProtKB/Swiss-Prot, Patented protein sequences, Protein Data Bank proteins, Metagenomic proteins, and Transcriptome Shotgun Assembly proteins. Using the databases listed above rBlast may be utilized to run protein BLAST to identify proteins with 100% identity to the human proteome.

In certain aspects, Varcode software is utilized in python to predict the impact of the genome variant data. Varcode generates the wild type and mutant protein sequences from the mutation and/or neoepitopes at the desired peptide size. In certain aspects, the desired peptide size is 3, 4, 5, 6, 7, 8, 9, 10, 11, 12, 13, 14, 15, 16, 17, 18, 19, 20 or more amino acids. In one aspect, the desired peptide size is 15 amino acids.

Varcode dependency is Pyensembl, which is a python access point to Ensembl reference genome data to generate peptides. In some embodiments, Varcode predicts the impact of each mutation on the protein. Silent mutations and mutations that are in noncoding regions on the genome are excluded. Varcode is then used to generate mutations across the peptide sequence. These variant peptides (i.e., candidate neoantigens) containing the mutations generated with Varcode are further analyzed with in silico methods and/or biochemical assays (e.g., a peptide-DNA conjugate assay) to identify which peptides bind patient-specific MHC class I and II proteins and which are ultimately be recognized by the patient's tumor infiltrating lymphocytes (TILs).

Analysis of Neoantigens with a Peptide-DNA Conjugate Approach

The peptide-DNA conjugate platform is well suited to rapidly screen large populations of diverse molecules for candidate neoantigens. In some embodiments, the present invention provides the following strategy for identification of neoantigens. According to various embodiments, a peptide-DNA conjugate method comprises a method for pooled, highly-parallel expression of proteins, each associated with a nucleic acid barcode. In some embodiments, the peptide-DNA conjugate method comprises a method for pooled, highly-parallel in vitro expression of proteins, each covalently linked to a DNA barcode through a puromycin-containing linkage. In other embodiments, the peptide-DNA conjugate method comprises phage display, mRNA display, or other method.

The peptide-DNA conjugate approach is a proprietary technology that can rapidly generate a large number of potential neoantigens as candidate binding agents for the patient-specific HLA proteins. The peptide-DNA conjugate approach is a method that generates peptide libraries (10-50 amino acids long) with each peptide conjugated to a unique DNA tag that can be used to monitor peptide abundance following binding experiments. Importantly, the peptide sequences can be used in large multiplexed binding assays with upwards of 100,000 of unique programmable peptides.

Binding assays for any molecular target can be used to screen large diverse peptide-DNA conjugate libraries. A biological structure (e.g., a patient-specific HLA proteins or fragments thereof corresponding to the patient's genotype) can be mixed with diverse peptides, separated from unbound peptides, and then queried for those that “stick.” The particular libraries could be intelligently designed libraries based upon prior knowledge of the target (e.g., from exome sequencing and analysis).

The peptide-DNA conjugate platform is described in greater detail in U.S. Pat. Nos. 9,958,454; 10,288,608; and U.S. Patent Application Publication No. 2016/0025726, the contents of which are hereby incorporated by reference.

In some embodiments, the peptide-DNA conjugate method is used to identifying neoantigens. In certain aspects, identification of neoantigen comprises (i) providing a library of peptide constructs, wherein each peptide construct of the library comprises a peptide portion and an identifying nucleic acid portion that identifies the peptide portion, and the peptide portion of at least one of the peptide constructs is capable of specific binding to the HLA proteins or fragments thereof; (ii) contacting the HLA proteins or fragments thereof with the library of peptide constructs; (iii) separating the at least one peptide construct comprising a peptide portion capable of specific binding to the HLA proteins or fragments thereof from peptide constructs comprising a peptide portion not capable of specific binding to the HLA proteins or fragments thereof; (iv) sequencing all or a portion of the identifying nucleic acid portion of the at least one peptide construct capable of specific binding to the HLA proteins or fragments thereof. In some aspects, the identifying nucleic acid portion of each peptide construct comprises a polynucleotide sequence or complement thereof encoding the peptide portion of the peptide construct.

Identification of Neoantigens with Phage Display, Ribosomal display, mRNA Display, Yeast Display, Bacterial Display, and Related Techniques

In some embodiments, the polypeptides of interest are genetically encoded to facilitate identification of neoantigens. An example of a genetically encoded polypeptide library is a mRNA display library. Another example is a replicable genetic display package (rgdp) library such as a phage display library. In one embodiment, the polypeptides of interest are genetically encoded as a phage display library. In these embodiments, the nucleic acid may be comprised by the phage genome. In these embodiments, the polypeptide may be comprised by the phage coat.

In some embodiments, the invention may be used to produce a genetically encoded combinatorial library of polypeptides which are generated by translating a number of nucleic acids into corresponding polypeptides and linking molecules of said molecular scaffold to said polypeptides. The genetically encoded combinatorial library of polypeptides may be generated by phage display, yeast display, ribosomal display, bacterial display or mRNA display.

### Phage Display

Techniques and methodology for performing phage display can be found in WO 2009/098450.

Different phage display systems have been developed throughout the years, making use of different phage vectors (M13 filamentous phage, lambda, T4 and T7 phage) and various phage coat proteins for covalent fusion. The M13 filamentous phage is employed most commonly. The strength of the filamentous phage display system lies within the lysogenic life cycle of this phage and the availability of M13 phagemid vectors (Webster, 1006; Hufton et al., 1999). Lysogenic phage integrate their DNA into the host cell genome, are replicated along with the bacterial cell and do not require the lysis of the bacterial cell for phage particle formation. Instead, phage particles are shed from the bacterial surface without inducing cell death (Webster, 1996). Moreover, the development of M13 phagemid vectors has allowed for excellent workability. Phagemids are plasmids containing the replication origin and packaging signal of the filamentous phage, together with the plasmid origin of replication and the gene encoding the phage coat protein coupled with the DNA insert (Webster, 1996; Armstrong et al., 1996). For phage propagation, bacterial cells infected with phagemid need to be “superinfected” with a so-called helper phage that provides all the other essential phage components for the formation of viable phage virions. Besides excellent workability, the use of a phagemid vector system allows for monovalent display of the recombinant protein (maximally one recombinant protein per phage virion) as the helper phage contributes non-recombinant phage coat proteins (Armstrong et al., 1996). Different M13 vector systems for phage display through various coat proteins are available (Smith and Petrenko, 1997; Barbas, 1993). Major coat protein pVIII and minor coat protein pIII, are used most frequently for display purposes (Armstrong et al., 1996; Rodi and Makowski, 1999). As the N-terminal end of both proteins is exposed at the phage surface, foreign DNA sequences are inserted upstream of the genes encoding the coat proteins. The development of phage vectors for C-terminal fusion to M13 minor coat protein pVI, of which the C-terminal end is exposed at the surface of the phage, has been an important step towards the development of cDNA phage display libraries (Hufton et al., 1999; Jespers et al., 1995).

More recently, display methods were also developed in the lytic phage systems, namely for lambda phage, T4 and T7 phage. For the formation and shedding of recombinant lytic phage virions containing recombinant coat proteins, bacterial cells need to be lysed on phage propagation (Russel, 1991). Moreover, as there are no plasmid vectors available for the lytic phage, DNA isolation and experimental approaches are more labor-intensive in comparison to working with plasmids (Sambrook et al., 1989). As both lytic and lysogenic phage life cycles employ different phage assembly strategies, both approaches allow the display of different proteins (Hufton et al., 1999). As the virion proteins of M13 filamentous phage (and thus, also the recombinant phage coat protein) are embedded into the bacterial cell membrane prior to phage virion assembly, this process puts constraints on the proteins that can be displayed at the surface of the phage; for efficient display, the cDNA products must be able to traverse the bacterial cell membrane and need to allow for the formation of a viable and infectious virion (Webster, 1996; Russel, 1991; Rodi et al., 2002). For lytic phage virion production on the other hand, the recombinant proteins are formed and retained within the cytosol of bacterial cells prior and during virion assembly so that the spectrum of recombinant proteins that can be displayed by lytic phage is less constrained (Hufton et al., 1999; Russel, 1991; Krumpe et al., 2006).

Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999)J Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993)EMBO J 12:725-734; Hawkins et al. (1992) J Mol Biol 226:889-896; Clackson et al. (1991) Nature 352:624-628; Gram et al. (1992) Proc Natl Acad Sci USA 89:3576-3580; Garrard et al. (1991)Bio/Technology 9:1373-1377; Rebar et al. (1996)Methods Enzymol. 267:129-49; Hoogenboom et al. (1991)Nuc Acid Res 19:4133-4137; and Barbas et al. (1991) PNAS 88:7978-7982.

### Ribosomal Display

In certain aspects, the methods described herein comprise the use of ribosomal display to identify neoantigens. Ribosomal display can be used to perform in vitro protein synthesis of a protein of interest (e.g., a candidate neoantigen). Ribosomal display can also be used to perform in vitro protein evolution to create proteins with desired properties, for example, Fab fragments that bind to a specific target molecule. Ribosomal display is a well-known technology useful for generating libraries. This entirely in vitro method allows for libraries with a diversity of 10¹⁶ members. The process results in translated proteins that are associated with their RNA progenitor which can be used, as a complex, to select for proteins with desired properties (e.g., bind to an immobilized target molecule). The RNA-protein complex that shows the desired property, e.g., high binding affinity, can then be reverse transcribed to cDNA and the sequence amplified via PCR. The process also provides for repeated cycles of iteration or protein expression. In addition, nucleic acid mutations can be introduced efficiently into the selected nucleic acid library in subsequent cycles, leading to continuous DNA diversification and thus, protein evolution. The end result is a nucleic acid sequence that can be used to produce a protein of interest (e.g., neoantigens that specifically bind to the patient-specific HLA proteins, and ultimately the patent's TILs).

In the method provided herein, a protein of interest is displayed on the surface of a ribosome from which the protein of interest is being translated. Briefly, a library of RNA molecules is translated in an in vitro translation system to the 3′ end of the RNA molecule, such that the ribosome does not fall off. This is accomplished by not incorporating a functional stop codon in the RNA template. Normally, the stop codon is recognized by release factors that trigger detachment of the nascent polypeptide from the ribosome. In ribosomal display, the peptide emerges from the ribosome, but does not fall free of the complex. This allows, for example, the nascent polypeptide to associate with another polypeptide to form a functional dimer. In some instances, there is an additional folding step in an oxidizing environment (important for forming disulfide bonds).

The whole complex of folded protein of interest, ribosome and RNA, which is stable for several days, can then be screened for desired properties, such as specific binding to a binding pair ligand by the translated protein of interest. The RNA encoding the selected protein of interest can be reverse transcribed into single stranded cDNA which can be converted to double-stranded DNA and amplified by PCR, providing the coding sequence of the selected protein (e.g., a neoantigen). The reverse transcription reaction can be performed on mRNA associated in the ribosomal display complex, or the mRNA can be isolated from the ribosomal display complex then used in the reverse transcription step. Suitable methods for disruption/dissociation of ribosome complexes are known in the art and include EDTA treatment and/or phenol extraction.

In general, nucleic acid (DNA) constructs for ribosomal display contain a promoter (T7, SP6 or T3), a translation initiation signal such as a Shine-Dalgarno (prokaryotic) or Kozak (eukaryotic) sequence, initiation codon, and coding sequence of the protein of interest (e.g., V_{H} or V_{L} chain domain). One or more nucleic acid sequences encoding one or more detection tags may be included to provide for production of a protein further comprising one or more detection tags (e.g., histidine tag). To enable the complete nascent protein to be displayed and fold into its active conformation, a spacer domain of at least 23-30 amino acids length can be added at the C terminus, to allow the protein to exit completely from the ribosome. The spacer can provide a known sequence for the design of primers for RT-PCR recovery of the DNA sequences.

To remove the stop codon from DNA, a 3′ primer lacking the stop codon can be used during PCR construction. Constructs designed for bacterial-based display systems can incorporate sequences containing stem-loop structures at the 5′ and 3′ ends of the DNA to stabilize mRNA against degradation by RNase activities in bacterial cell-free systems.

The mRNA translation system used in the methods described herein may be any suitable available system. A prokaryotic or eukaryotic translation system may be used, for example crude E. coli lysate (commercially available from e.g. Promega Corp., Madison, Wis.; Agilent Technologies, Santa Clara, Calif; GE Healthcare Biosciences, Pittsburgh, Pa.; Life Technologies, Carlsbad, Calif.), a reconstituted ribosome system such as PURE (see, e.g., Shimizu et al., Nat. Biotechnol., 19:751-755 (2001)), or a cell free protein synthesis system as described below.

The PURE system can include about 32 individually purified components used for in vitro protein biosynthesis (e.g., initiation, elongation and termination). In some embodiments, the components include initiation factors (e.g., IF1, IF2, IF3), elongation factors (e.g., EF-G, EF-Tu, EF-Ts), release factors (e.g., RF1, RF3), a termination factor (e.g., RRF), 20 aminoacyl-tRNA synthetases, methionyl-tRNA transformylase, T7 RNA polymerase, ribosomes, 46 tRNAs, NTPs, creatine phosphate, 10-formyl-5,6,7,8-tetrahydrofolic acid, 20 amino acids, creatine kinase, myokinase, nucleoside-diphosphate kinase and pyrophosphatase.

Ribosomal display has been used to successfully generate antibody fragments with high affinity for their target. Detailed description of ribosomal display is found in, e.g., Hanes, J. Proc. Natl. Acad. Sci. USA, 95: 14130-14135 (1998); Schaffitzel et al., J. Immunological Methods, 231:119-135 (1999); He et al., J. Immunological Methods, 231, 105-117 (1999); Roberts R W, Current Opinion in Chemical Biology, 3: 268-273 (1999).

### Other Display Methods for In Vitro Selection

Other in vitro library display methods can be used in the methods described herein, such as, but not limited to, mRNA display, bicistronic display, P2A display, and CIS display (cis-activity display).

mRNA Display

In mRNA display, each member of the RNA library is directly attached to the protein of interest it encodes by a stable covalent linkage to puromycin, an antibiotic that can mimic the aminoacyl end of tRNA (see, e.g., Robert R W and Szostak J W, Proc. Natl. Acad. Sci. USA, 94:12297-12302 (1997)). Puromycin is an aminonucleoside antibiotic, active in either prokaryotes or eukaryotes, derived from Streptomyces alboniger. Protein synthesis is inhibited by premature chain termination during translation taking place in the ribosome. Part of the molecule acts as an analog of the 3′ end of a tyrosyl-tRNA, where a part of its structure mimics a molecule of adenosine and another part mimics a molecule of tyrosine. It enters the A site and transfers to the growing chain, causing the formation of a puromycylated nascent chain and premature chain release. The 3′ position contains an amide linkage instead of the normal ester linkage of tRNA, making the molecule much more resistant to hydrolysis and stopping procession along the ribosome.

Other puromycin analog inhibitors of protein synthesis include O-demethylpuromycin, O-propargyl-puromycin, 9-{3′-deoxy-3′-[(4-methyl-L-phenylalanyl)amino]-o-D-ribofuranosyl}-6-(N,N′-dimethylamino)purine [L-(4-Me)-Phe-PANS] and 6-dimethylamino-9-[3-(p-azido-L-beta-phenylalanylamino)-3-deoxy-beta-ribofuranosyl]purine.

Members of the RNA library can be ligated to puromycin via a linker such as, but not limited to, a polynucleotide or a chemical linker, e.g., polyethylene glycol (Fukuda et al., Nucleic Acid Research, 34(19):e127 (2006)). In some embodiments, the polynucleotide linker comprising RNA is linked at the 3′ terminal end to puromycin. In other embodiments, the PEG liker is linked to puromycin.

As puromycin that is linked to the 3′ terminal end of a RNA molecule enters a ribosome, it establishes a covalent bond to the nascent protein (encoded by the RNA molecule) as a result 10 of peptidyl transferase activity in the ribosome. In turn, a stable amide linkage forms between the protein and the O-methyl tyrosine portion of puromycin.

The RNA library of RNA-puromycin fusions can undergo in vitro translation, as described herein, to generate RNA-puromycin-protein complexes (e.g., RNA-puromycin-neoantigen complexes). In some embodiments, the RNA-puromycin fusions comprise RNA molecules encoding candidate neoantigens identified by exome analysis of tumor cells and wild-type cells from a patient.

Affinity selection can be performed on the mRNA-displayed protein library to screen for proteins having desired properties, such a specific binding to a binding pair ligand. The mRNA display can be performed in solution or on a solid support. The selected mRNA-displayed protein of interest can be purified by standard methods known in the art such as affinity chromatography. The mRNA can be cloned, PCR amplified, and/or sequenced to determine the coding sequence of the selected protein of interest. In one embodiment, the selected mRNA-displayed library contains a population of neoantigens that bind to the patient-specific HLA proteins or fragments thereof, and ultimately the patient's TILs.

In some aspects, members of a library of nucleic acid members are linked to puromycin, where each member encodes a prospective protein of interest having a primary amino acid sequence different from the other proteins encoded by the other nucleic acid members. The mRNA display system can contain a population or mixture of different complexes such that each complex has a different mRNA linked to puromycin, the ribosome, and a prospective protein of interest.

### Biscistronic DNA Display

Bicistronic DNA display can be employed for in vitro selection of proteins of interest (see, e.g., Sumida et al., Nucleic Acid Research, 37(22):e147 (2009)). The method is based on complexing in vitro translated proteins of interest to their encoding DNA, which used to determine the sequence of the protein of interest. Typically, a DNA template containing multiple ORFs that can be linked to the protein it encodes is generated. In addition, the coupled transcription/translation reaction is compartmentalized in a water-in-oil emulsion (e.g., micelle).

In some instances, when multiple ORFs are used, they can be separated by ribosomal binding sides. In some embodiments, the coding sequence of the protein of interest is fused to the coding sequence of streptavidin. In some embodiments, the DNA template is biotinylated through a linker. The linker can be cleavable.

During in vitro transcription/translation, the protein can be expressed in a micelle. In one non-limiting illustrative embodiment, the DNA template contains the coding sequence of a candidate neoantigen, and the candidate neoantigen is expressed in a micelle. In embodiments, a candidate neoantigen forms and is associated with the DNA encoding the candidate neoantigen through a linkage, such as a streptavidin-biotin link. The DNA displayed protein of interest, such as a candidate neoantigen, can be recovered from the emulsion and screened by affinity selection. The DNA template of the selected DNA-displayed protein of interest can be cleaved from the complex by methods such as UV irradiation and then PCR amplified, cloned, and/or sequenced.

In some embodiments, the methods provided herein include generating a library of nucleic acid members, each member encoding a prospective protein of interest (i.e., a candidate neoantigen) having a primary amino acid different from the other proteins encoded by the other nucleic acid members. In some embodiments, the library comprises DNA members, and the method includes transcribing the library into RNA and translating the RNA in a cell free protein synthesis system to generate a bicistronic DNA display system in a water-in-oil emulsion. In some embodiments, the bicistronic DNA display system contains a population of proteins of interest that are selected for specific binding to a binding pair ligand. In one embodiment, the bicistronic DNA display system contains a population of candidate neoantigens, wherein each candidate neoantigen is associated with a DNA molecule encoding the candidate neoantigen.

### P2A DNA Display

In P2A DNA display, which utilizes the cis-activity of the endonuclease P2A, a fusion protein of P2A and a protein of interest binds to the same DNA molecule from which it is expressed (see, e.g., Reiersen et al., Nucleic Acids Research, 33(1): e10). The DNA template can contain the coding sequence encoding the protein of interest genetically fused to the coding sequence of P2A, a promoter, and an origin of replication. The coding sequence of P2A can be obtained and a genetic fusion with the protein of interest can be constructed by standard methods known to those in the art.

In some embodiments, P2A DNA display is used to select a protein of interest. The protein of interest can be selected by generating a library of fusion proteins, where each member of the library comprises a fusion protein between P2A and a protein of interest, and selecting proteins of interest based on a desired property, for example, specific binding to a binding pair ligand. The library can be constructed from a library of nucleic acid members, where each member comprises a DNA template encoding a different protein of interest (i.e., each protein of interest has a primary amino acid sequence that is different from other proteins encoded by other members of the nucleic acid library) that is genetically fused to the coding sequence of P2A.

In some embodiments, the P2A DNA-displayed library can be screened by affinity selection strategies, e.g., in solution or on a solid support. The selected protein of interest can be purified by, e.g., affinity chromatography, and the complexed DNA can be PCR amplified, cloned and/or sequenced.

### CIS Display

Similar to P2A DNA display, CIS display involves a DNA-based approach to directly link in vitro transcribed/translated proteins to the DNA molecules that encode them (see, e.g., Odegrip et al., Proc. Natl. Acad. Sci. USA, 101(9):2806-2810). This method uses RepA, a DNA replication initiator protein, to non-covalently bind to the DNA molecule from which it is expressed if the DNA molecule has a CIS element. The DNA molecule can be created to encode proteins of interest (e.g., candidate neoantigens) in addition to RepA.

In some embodiments, CIS display is used to select a protein of interest. The protein of interest can be selected by generating a DNA library of fusion proteins, where each member comprises a fusion protein between RepA and a protein of interest, and selecting proteins of interest based on a desired property, for example, specific binding to a binding pair ligand. The library can be constructed from a DNA library, where each member of the library comprises a DNA template encoding a different protein of interest (i.e., each protein of interest has a primary amino acid sequence that is different from other proteins encoded by other members of the nucleic acid library) that is genetically fused to the coding sequence of RepA.

In some embodiments, members of the DNA library contain a coding sequence of the protein of interest, a coding sequence of RepA, a CIS element, an origin of replication and a promoter, wherein the coding sequence of the protein of interest is genetically fused to the coding sequence of RepA. The CIS element can be genetically linked to the RepA coding sequence.

In some embodiments, the library contains a fusion protein between RepA and a protein of interest, and a DNA molecule encoding the fusion protein.

The RepA DNA-displayed library can be screened by affinity selection strategies, e.g., in solution or on a solid support. The selected protein of interest can be purified by, e.g., affinity chromatography, and the complexed DNA can be PCR amplified, cloned and/or sequenced.

### Cell-Free Protein Synthesis (CFPS)

In order to express the biologically active proteins of interest described herein (i.e., neoantigens), a cell free protein synthesis system can be used. Cell extracts have been developed that support the synthesis of proteins in vitro from purified mRNA transcripts or from mRNA transcribed from DNA during the in vitro synthesis reaction.

CFPS of polypeptides in a reaction mix comprises bacterial extracts and/or defined reagents. The reaction mix comprises at least ATP or an energy source; a template for production of the macromolecule, e.g., DNA, mRNA, etc.; amino acids, and such co-factors, enzymes and other reagents that are necessary for polypeptide synthesis, e.g., ribosomes, tRNA, polymerases, transcriptional factors, aminoacyl synthetases, elongation factors, initiation factors, etc. In one embodiment of the invention, the energy source is a homeostatic energy source. Also included may be enzyme(s) that catalyze the regeneration of ATP from high-energy phosphate bonds, e.g., acetate kinase, creatine kinase, etc. Such enzymes may be present in the extracts used for translation, or may be added to the reaction mix. Such synthetic reaction systems are well-known in the art, and have been described in the literature.

The templates for cell-free protein synthesis can be either mRNA or DNA. The template can comprise sequences for any particular gene of interest, and may encode a full-length polypeptide or a fragment of any length thereof. Nucleic acids that serve as protein synthesis templates are optionally derived from a natural source or they can be synthetic or recombinant. For example, DNAs can be recombinant DNAs, e.g., plasmids, viruses or the like.

The term “reaction mix” as used herein, refers to a reaction mixture capable of catalyzing the synthesis of polypeptides from a nucleic acid template. The reaction mixture comprises extracts from bacterial cells, e.g., E. coli S30 extracts. S30 extracts are well known in the art, and are described in, e.g., Lesley, S. A., et al. (1991), J. Biol. Chem. 266, 2632-8.

The synthesis can be performed under either aerobic or anaerobic conditions.

In some embodiments, the bacterial extract is dried. The dried bacterial extract can be reconstituted in milli-Q water (e.g., reverse osmosis water) at 110% of the original solids as determined by measuring the percent solids of the starting material. In one embodiment, an accurately weighed aliquot of dried extract, representing 110% of the original solids of 10 mL of extract, is added to 10 mL of Milli-Q water in a glass beaker with a stir bar on a magnetic stirrer. The resulting mixture is stirred until the powder is dissolved. Once dissolved, the material is transferred to a 15 mL Falcon tube and stored at −80 C unless used immediately.

The volume percent of extract in the reaction mix will vary, where the extract is usually at least about 10% of the total volume; more usually at least about 20%; and in some instances, may provide for additional benefit when provided at least about 50%; or at least about 60%; and usually not more than about 75% of the total volume.

The general system includes a nucleic acid template that encodes a protein of interest. The nucleic acid template is an RNA molecule (e.g., mRNA) or a nucleic acid that encodes an mRNA (e.g., RNA, DNA) and be in any form (e.g., linear, circular, supercoiled, single stranded, double stranded, etc.). Nucleic acid templates guide production of the desired protein.

To maintain the template, cells that are used to produce the extract can be selected for reduction, substantial reduction or elimination of activities of detrimental enzymes or for enzymes with modified activity. Bacterial cells with modified nuclease or phosphatase activity (e.g., with at least one mutated phosphatase or nuclease gene or combinations thereof) can be used for synthesis of cell extracts to increase synthesis efficiency. For example, an E. coli strain used to make an S30 extract for CFPS can be RNase E or RNase A deficient (for example, by mutation).

In a generic CFPS reaction, a gene encoding a protein of interest is expressed in a transcription buffer, resulting in mRNA that is translated into the protein of interest in a CFPS extract and a translation buffer. The transcription buffer, cell-free extract and translation buffer can be added separately, or two or more of these solutions can be combined before their addition, or added contemporaneously.

To synthesize a protein of interest in vitro, a CFPS extract at some point comprises a mRNA molecule that encodes the protein of interest. In some CFPS systems, mRNA is added exogenously after being purified from natural sources or prepared synthetically in vitro from cloned DNA using RNA polymerases such as RNA polymerase II, SP6 RNA polymerase, T3 RNA polymerase, T7 RNA polymerase, RNA polymerase III and/or phage derived RNA polymerases. In other systems, the mRNA is produced in vitro from a template DNA; both transcription and translation occur in this type of CFPS reaction. In some embodiments, the transcription and translation systems are coupled or comprise complementary transcription and translation systems, which carry out the synthesis of both RNA and protein in the same reaction. In such in vitro transcription and translation systems, the CFPS extracts contain all the components (exogenous or endogenous) necessary both for transcription (to produce mRNA) and for translation (to synthesize protein) in a single system.

A cell free protein synthesis reaction mixture comprises the following components: a template nucleic acid, such as DNA, that comprises a gene of interest operably linked to at least one promoter and, optionally, one or more other regulatory sequences (e.g., a cloning or expression vector containing the gene of interest) or a PCR fragment; an RNA polymerase that recognizes the promoter(s) to which the gene of interest is operably linked and, optionally, one or more transcription factors directed to an optional regulatory sequence to which the template nucleic acid is operably linked; ribonucleotide triphosphates (rNTPs); optionally, other transcription factors and co-factors therefor; ribosomes; transfer RNA (tRNA); other or optional translation factors (e.g., translation initiation, elongation and termination factors) and co-factors therefore; one or more energy sources, (e.g., ATP, GTP); optionally, one or more energy regenerating components (e.g., PEP/pyruvate kinase, AP/acetate kinase or creatine phosphate/creatine kinase); optionally factors that enhance yield and/or efficiency (e.g., nucleases, nuclease inhibitors, protein stabilizers, chaperones) and co-factors therefore; and; optionally, solubilizing agents. The reaction mix further comprises amino acids and other materials specifically required for protein synthesis, including salts (e.g., potassium, magnesium, ammonium, and manganese salts of acetic acid, glutamic acid, or sulfuric acids), polymeric compounds (e.g., polyethylene glycol, dextran, diethyl aminoethyl dextran, quaternary aminoethyl and aminoethyl dextran, etc.), cyclic AMP, inhibitors of protein or nucleic acid degrading enzymes, inhibitors or regulators of protein synthesis, oxidation/reduction adjuster (e.g., DTT, ascorbic acid, glutathione, and/or their oxides), non-denaturing surfactants (e.g., Triton X-100), buffer components, spermine, spermidine, putrescine, etc. Components of CFPS reactions are discussed in more detail in U.S. Pat. Nos. 7,338,789 and 7,351,563, and U.S. App. Pub. No. 2010/0184135, the disclosures of which are incorporated by reference in its entirety for all purposes.

Depending on the specific enzymes present in the extract, for example, one or more of the many known nuclease, polymerase or phosphatase inhibitors can be selected and advantageously used to improve synthesis efficiency.

Protein and nucleic acid synthesis typically requires an energy source. Energy is required for initiation of transcription to produce mRNA (e.g., when a DNA template is used and for initiation of translation high energy phosphate for example in the form of GTP is used). Each subsequent step of one codon by the ribosome (three nucleotides; one amino acid) requires hydrolysis of an additional GTP to GDP. ATP is also typically required. For an amino acid to be polymerized during protein synthesis, it must first be activated. Significant quantities of energy from high energy phosphate bonds are thus required for protein and/or nucleic acid synthesis to proceed.

An energy source is a chemical substrate that can be enzymatically processed to provide energy to achieve desired chemical reactions. Energy sources that allow release of energy for synthesis by cleavage of high-energy phosphate bonds such as those found in nucleoside triphosphates, e.g., ATP, are commonly used. Any source convertible to high energy phosphate bonds is especially suitable. ATP, GTP, and other triphosphates can normally be considered as equivalent energy sources for supporting protein synthesis.

To provide energy for the synthesis reaction, the system can include added energy sources such as glucose, pyruvate, phosphoenolpyruvate (PEP), carbamoyl phosphate, acetyl phosphate, creatine phosphate, phosphopyruvate, glyceraldehyde-3-phosphate, 3-Phosphoglycerate and glucose-6-phosphate that can generate or regenerate high-energy triphosphate compounds such as ATP, GTP, other NTPs, etc.

When sufficient energy is not initially present in the synthesis system, an additional source of energy is preferably supplemented. Energy sources can also be added or supplemented during the in vitro synthesis reaction.

In some embodiments, the cell-free protein synthesis reaction is performed using the PANOx-SP system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, phosphoenol pyruvate (PEP), NAD, CoA, Na⁺ oxalate, putrescine, spermidine, and S30 extract.

In some instances, the cell-free synthesis reaction does not require the addition of commonly secondary energy sources, yet uses co-activation of oxidative phosphorylation and protein synthesis. In some instances, CFPS is performed in a reaction such as the Cytomim (cytoplasm mimic) system. The Cytomim system is defined as a reaction condition performed in the absence of polyethylene glycol with optimized magnesium concentration. This system does not accumulate phosphate, which is known to inhibit protein synthesis.

The presence of an active oxidative phosphorylation pathway can be tested using inhibitors that specifically inhibit the steps in the pathway, such as electron transport chain inhibitors. Examples of inhibitors of the oxidative phosphorylation pathway include toxins such as cyanide, carbon monoxide, azide, carbonyl cyanide m-chlorophenyl hydrazone (CCCP), and 2,4-dinitrophenol, antibiotics such as oligomycin, pesticides such as rotenone, and competitive inhibitors of succinate dehydrogenase such as malonate and oxaloacetate.

In some embodiments, the cell-free protein synthesis reaction is performed using the Cytomim system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, sodium pyruvate, NAD, CoA, Na⁺ oxalate, putrescine, spermidine, and S30 extract. In some embodiments, the energy substrate for the Cytomim system is pyruvate, glutamic acid, and/or glucose. In some embodiments of the system, the nucleoside triphosphates (NTPs) are replaced with nucleoside monophosphates (NMPs).

The cell extract can be treated with iodoacetamide in order to inactivate enzymes that can reduce disulfide bonds and impair proper protein folding. In some embodiments, the cell extract comprises an exogenous protein chaperone. The protein chaperone can be expressed by the bacterial strain used to make the cell free extract, or the protein chaperone can be added to the cell extract. Non-limiting examples of exogenous protein chaperones include disulfide bond isomerase (PDI), such as, but not limited to E. coli DsbC, and peptidyl prolyl cis-trans isomerase (PPIase), such as but not limited to FkpA. In some embodiments, the extract comprises both a PDI and a PPIase, e.g., both DsbC and FkpA. Glutathione disulfide (GSSG) and/or glutathione (GSH) can also be added to the extract at a ratio that promotes proper protein folding and prevents the formation of aberrant protein disulfides.

In some embodiments, the CFPS reaction includes inverted membrane vesicles to perform oxidative phosphorylation. These vesicles can be formed during the high pressure homogenization step of the preparation of cell extract process, as described herein, and remain in the extract used in the reaction mix.

Methods of preparing a cell extract are described in, e.g., Zawada, J. “Preparation and Testing of E. coli S30 In Vitro Transcription Translation Extracts”, Douthwaite, J. A. and Jackson, R. H. (eds.), Ribosomal display and Related Technologies: Methods and Protocols, Methods in Molecular Biology, vol. 805, pp. 31-41 (Humana Press, 2012); Jewett et al., Molecular Systems Biology: 4, 1-10 (2008); Shin J. and Norieaux V., J Biol. Eng., 4:8 (2010). Briefly, a bacterial culture is grown and harvested; suspended in an appropriate buffer (e.g., S30 buffer), and homogenized to lyse the cells.

The cell-free extract can be thawed to room temperature before use in the CFPS reaction. The extract can be incubated with 50 μM iodoacetamide for 30 minutes when synthesizing protein with disulfide bonds. In some embodiments, the CFPS reaction includes about 30% (v/v) iodoacetamide-treated extract with about 8 mM magnesium glutamate, about 10 mM ammonium glutamate, about 130 mM potassium glutamate, about 35 mM sodium pyruvate, about 1.2 mM AMP, about 0.86 mM each of GMP, UMP, and CMP, about 2 mM amino acids (about 1 mM for tyrosine), about 4 mM sodium oxalate, about 0.5 mM putrescine, about 1.5 mM spermidine, about 16.7 mM potassium phosphate, about 100 mM T7 RNA polymerase, about 2-10 μg/mL plasmid DNA template, about 1-10 μM E. coli DsbC, and a total concentration of about 2 mM oxidized (GSSG) glutathione. Optionally, the cell free extract can include 1 mM of reduced (GSH).

### Generating a Lysate

The methods and systems described herein can use a cell lysate for in vitro translation of a target protein of interest. For convenience, the organism used as a source for the lysate may be referred to as the source organism or host cell. Host cells may be bacteria, yeast, mammalian or plant cells, or any other type of cell capable of protein synthesis. A lysate comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2, and IF-3, elongation factors such as EF-Tu, EF-Ts, and EF-G, release factors such as RF-1, RF-2, and RF-3, and the like.

An embodiment uses a bacterial cell from which a lysate is derived. A bacterial lysate derived from any strain of bacteria can be used in the methods of the invention. The bacterial lysate can be obtained as follows. The bacteria of choice are grown to log phase in any of a number of growth media and under growth conditions that are well known in the art and easily optimized by a practitioner for growth of the particular bacteria. For example, a natural environment for synthesis utilizes cell lysates derived from bacterial cells grown in medium containing glucose and phosphate, where the glucose is present at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients. Cells that have been harvested overnight can be lysed by suspending the cell pellet in a suitable cell suspension buffer, and disrupting the suspended cells by sonication, breaking the suspended cells in a French press, continuous flow high pressure homogenization, or any other method known in the art useful for efficient cell lysis. The cell lysate is then centrifuged or filtered to remove large DNA fragments and cell debris.

The bacterial strain used to make the cell lysate generally has reduced nuclease and/or phosphatase activity to increase cell free synthesis efficiency. For example, the bacterial strain used to make the cell free extract can have mutations in the genes encoding the nucleases RNase E and RNase A. The strain may also have mutations to stabilize components of the cell synthesis reaction such as deletions in genes such as tnaA, speA, sdaA or gshA, which prevent degradation of the amino acids tryptophan, arginine, serine and cysteine, respectively, in a cell-free synthesis reaction. Additionally, the strain may have mutations to stabilize the protein products of cell-free synthesis such as knockouts in the proteases ompT or lonP.

DNA Expression Cassette without Stop Codon

In some embodiments, the nucleic acid template used in the ribosomal display reaction system comprises a DNA expression cassette that is capable of expressing an RNA encoding the protein of interest, where the RNA lacks an operable stop codon. To remove the stop codon from the protein coding region in the expression cassette, a PCR primer lacking the stop codon can be used to amplify the coding region for the protein of interest, such that the entire coding region from the translation start to the sequence encoding the C-terminal amino acid is amplified.

MHC Binding Assay with Neoantigen Library

The major histocompatibility complex (MHC) is a collection of genes encoding glycoproteins called MHC proteins. The primary function of an MHC protein in vivo is to present antigen in a form capable of being recognized by a TCR. An MHC protein is bound to an antigen in the form of an antigenic peptide to form an MHC-peptide complex.

MHC proteins, also known as human leukocyte antigen (HLA) in humans and H-2 region in mice, are classified in two categories: class I and class II MHC proteins. These proteins are comprised of a cluster of highly polymorphic genes. Specifically, human HLA-A, HLA-B, and HLA-C are known as class I MHC molecules, whereas human HLA-DP, HLA-DQ and HLA-DR are known as class II MHC molecules. The HLA loci include HLA-DP, HLA-DN, HLA-DM, HLA-DO, HLA-DQ, HLA-DR, HLA-A, HLA-B and HLA-C. Each of these loci contains different alleles in the human population. The different subtypes encoded by these allelic variants are intended to be within the scope of the invention. An MHC class II protein is a heterodimeric integral membrane protein comprising one α and one β chain in noncovalent association. The a chain has two extracellular domains (α₁ and α₂), and a transmembrane (TM) and a cytoplasmic (CYT) domain. The β chain contains two extracellular domains (β₁ and β₂), and a TM and CYT domain. An MHC class I protein is an integral membrane protein comprising a glycoprotein heavy chain having three extracellular domains (i.e., α₁, α₂ and α₃), and a TM and CYT domain. The heavy chain is noncovalently associated with a soluble subunit called β₂-microglobulin (β₂ₘ).

In some embodiments, the binding assay (e.g., the MHC-peptide-DNA conjugate assay) of the present invention employs the use of a spaceholder molecule linked to the MHC class II component, e.g., the R chain, by a processable linker. A spaceholder molecule of the present invention may be any peptide that is capable of binding to a peptide binding groove of an MHC protein in such a manner that binding of any other peptide in the peptide binding groove is hindered. Preferably, the spaceholder molecule binds within the peptide binding groove with intermediate affinity, and more preferably with low affinity, at approximately neutral pH. In one embodiment, the length of a spaceholder molecule extends from about 5 to about 40 amino acid residues, more preferably from about 6 to about 30 amino acid residues, 8 to about 20 amino acid residues and even more preferably from about 12 to 15 amino acids residues. In a further embodiment, a spaceholder molecule is about 13 amino acids residues. Examples of suitable spaceholder molecules include, without limitation, (in single letter amino acid code) PVSKMRMATPLLMQA (SEQ ID NO: 25), also known as CLIP; AAMAAAAAAAMAA (SEQ ID NO: 26); AAMAAAAAAAAAA (SEQ ID NO: 27); AAFAAAAAAAAAA (SEQ ID NO: 28); ASMSAASAASMAA (SEQ ID NO: 29), and functional equivalents thereof. In one embodiment, the spaceholder molecule will have the consensus sequence AAXAAAAAAAXAA (SEQ ID NO: 30), wherein X is any amino acid. The ability of maintaining a spaceholder molecule within the binding groove of the MHC class II molecule prevents “empty” molecules from forming. The formation of “empty” MHC class II molecules has been a major limiting factor due to the tendency of these “empty” molecules to aggregate, thus making isolation of functional MHC class II components difficult.

The spaceholder molecule of the present invention may be covalently linked to the MHC molecule by a linker having an amino acid sequence that contains a target site for an enzyme capable of cleaving proteins. Such linkers are referred to herein as “processable linkers”. Examples of processable linkers of the present invention include linkers containing target sites for enzymes such as collagenases, metalloproteases, serine proteases, cysteine proteases, kallikriens, thrombin, and plasminogen activators. A preferred processable linker of the present invention includes a linker having a thrombin cleavage site.

Suitable linkers useful in the present invention can also be designed using various methods. For example, X-ray crystallographic data of an MHC protein can be used to design a linker of suitable length and charge such that the linker does not interfere with the binding of the spaceholder molecule to the peptide binding groove of the MHC class II component. Such methods are included in the present invention.

The length of a linker of the present invention is preferably sufficiently short (i.e., small enough in size) such that the linker does not substantially inhibit binding between the spaceholder molecule and the MHC class II component. The length of a linker of the present invention may range from about 1 amino acid residue to about 40 amino acid residues, more preferably from about 5 amino acid residues to about 30 amino acid residues, and even more preferably from about 8 amino acid residues to about 20 amino acid residues.

The cleavage of the linker facilitates the release of the spaceholder molecule thereby freeing the peptide binding groove. The MHC class II component may then be incubated with the library of candidate neoantigens (e.g., a peptide-DNA conjugate library of peptides designed from exome analysis of tumor and wild-type cells) to facilitate the binding of the antigen peptide molecule to the MHC class II component. After sufficient time to allow binding, which can be readily determined by one skilled in the art, the MHC class II component which has bound to the antigen peptide molecule is recovered.

In certain embodiments, the step of cleaving the linker and incubating with the antigen peptide molecule is repeated using different antigen peptide molecules. The advantage of repeating these steps is to allow for the formation of a number of MHC class II components that recognize several antigenic epitopes. Furthermore, these steps can be carried out using MHC class II molecules constructed with varying allelic forms of the MHC class II genes. This feature of the assay therefore allows for the generation of several MHC class II components which are specific for a number of different MHC allelic genotypes.

Alternatively, the binding assay (e.g., the MHC-peptide-DNA conjugate assay) of the present invention employs a phage display system (see, for example, Hammer J et al., Promiscuous and allele-specific anchors in HLA-DR-binding peptides, Cell (1993) 74(1):197-203). MHC II components are expressed with phage display. The phages expressing the MHC II components are then incubated with the library of candidate neoantigens (e.g., a peptide-DNA conjugate library of peptides designed from exome analysis of tumor and wild-type cells) to facilitate the binding of the antigen peptide molecule to the MHC class II component. After sufficient time to allow binding, which can be readily determined by one skilled in the art, the MHC class II component which has bound to the antigen peptide molecule is recovered.

In certain aspects, an in silico analysis is used to determine specific binding between neoantigens and MHC class I proteins or fragments thereof. The in silico analysis comprises applying a computational algorithm to predict relative binding to MHC I proteins based on the peptide sequences of the neoantigens. Tools such as netMHCpan are used for prediction of binding of peptides/neoantigens to the MHC proteins (see Jurtz et al, NetMHCpan-4.0: Improved Peptide-MHC Class I Interaction Predictions Integrating Eluted Ligand and Peptide Binding Affinity Data, J Immunol (2017) 199(9):3360-3368). The input to this analysis is the peptide sequences and the MHC alleles of interest, and the output is a predicted binding affinity.

In certain aspects, neoantigens demonstrating binding with MHC class I molecules or MHC class II molecules in an MHC binding assay or in silico are pooled to form a peptide mixture. This peptide mixture or oligoneucleotides encoding for the peptides from the peptide mixture can then be administered directly to the patient.

### Vaccine Administration

The administration of the peptides or oligonucleotides according to the methods provided herein can be carried out in any suitable manner for administering peptides or oligonucleotides, including but not limited to injection, transfusion, subcutaneously, intradermally, intratumorally, intranodally, intramedullary, intramuscularly, intrathecally, by intravenous or intralymphatic injection, or intraperitoneally. In some embodiments, the neoantigens or oligonucleotides are administered into a cavity formed by the resection of tumor tissue (i.e. intracavity delivery) or directly into a tumor prior to resection (i.e. intratumoral delivery). In one embodiment, the peptides or oligonucleotides are administered by intravenous injection.

In an embodiment, the invention includes a method of treating a cancer with one or more neoantigen peptide or with one or more oligonucleotide each having a nucleic acid sequence that encodes a neoantigen peptide, wherein a patient is pre-treated with non-myeloablative chemotherapy prior to the neoantigens treatment or oligonucleotide treatment according to the invention.

### Methods of Treating Cancer

The cancer treated by the disclosed compositions and methods can be any solid tumor. The cancer can also be metastatic and/or recurrent. Non-limiting examples of cancers include acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the vulva, chronic lymphocytic leukemia, chronic myeloid cancer, cervical cancer, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, peritoneum, omentum, and mesentery cancer, pharynx cancer, prostate cancer, rectal cancer, renal cancer, skin cancer, soft tissue cancer, testicular cancer, thyroid cancer, ureter cancer, urinary bladder cancer, and digestive tract cancer such as, e.g., esophageal cancer, gastric cancer, pancreatic cancer, stomach cancer, small intestine cancer, gastrointestinal carcinoid tumor, cancer of the oral cavity, colorectal cancer, and hepatobiliary cancer.

Triple Negative Breast Cancers (TNBCs), representing about 15% of all breast cancers, are highly aggressive type of tumors that lack estrogen receptor (ER), progesterone receptor (PR), and ERBB2 (HER2) gene amplification. TNBCs do not respond to hormonal therapy such as tamoxifen or aromatase inhibitors or therapies that target HER2 receptors, such as Herceptin (trastuzumab). Because of limited targets that are available for TNBCs, currently there is an intense interest in finding new targets and thus personalized medications that can treat this type of breast cancer. Therefore, in some embodiments, the cancer is a triple negative breast cancer (TNBC).

### Combination Cancer Therapies

The disclosed compositions and methods can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

Numerous anti-cancer drugs are available for combination with the present method and compositions. The following is a non-exhaustive lists of anti-cancer (anti-neoplastic) drugs that can be used in conjunction with or without irradiation: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

The disclosed compositions and methods can be used in combination with one or more immune checkpoint inhibitors. By immune checkpoint inhibitor it is meant a compound that inhibits a protein in the checkpoint signally pathway. Proteins in the checkpoint signally pathway include for example, CD27, CD28, CD40, CD 122, CD137, OX40, GITR, ICOS, A2AR, B7-H3, B7-H4, BTLA, CTLA-4, IDO, KIR, LAG3, PD-1, PD-L1, PD-L2, TIM-3, TIGIT, Lairl, CD244, HAVCR2, CD200, CD200R1, CD200R2, CD200R4, LILRB4, PILRA, ICOSL, 4-1BB or VISTA. Immune checkpoint inhibitors are known in the art.

For example, the immune checkpoint inhibitor can be a small molecule. Small molecules can be, e.g., nucleic acids, peptides, polypeptides, peptidomimetics, carbohydrates, lipids or other organic or inorganic molecules.

Alternatively, the immune checkpoint inhibitor is an antibody or fragment thereof. For example, the antibody or fragment thereof is specific to a protein in the checkpoint signaling pathway, such as CD27, CD28, CD40, CD 122, CD137, OX40, GITR, ICOS, A2AR, B7-H3, B7-H4, BTLA, CTLA-4, IDO, KIR, LAG3, PD-1, PD-L1, PD-L2, TIM-3, TIGIT, Lairl, CD244, HAVCR2, CD200, CD200R1, CD200R2, CD200R4, LILRB4, PILRA, ICOSL, 4-1BB or VISTA.

Exemplary, anti-immune checkpoint antibodies include for example ipiliumab (anti-CTLA-4), penbrolizumab (anti-PD-L1), nivolumab (anti-PD-L1), atezolizumab (anti-PD-L1), and duralumab (anti-PD-L1).

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Example 1. Overview of Neoantigen Vaccines

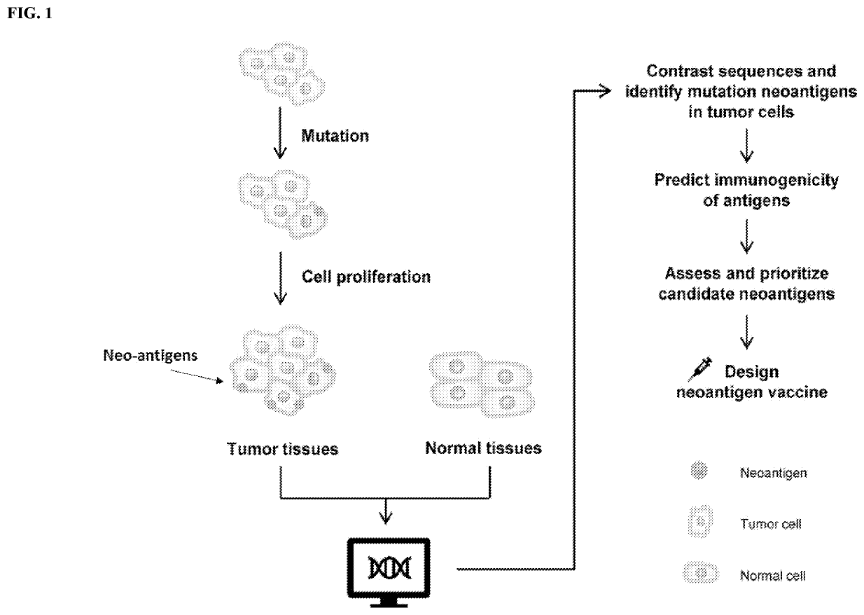

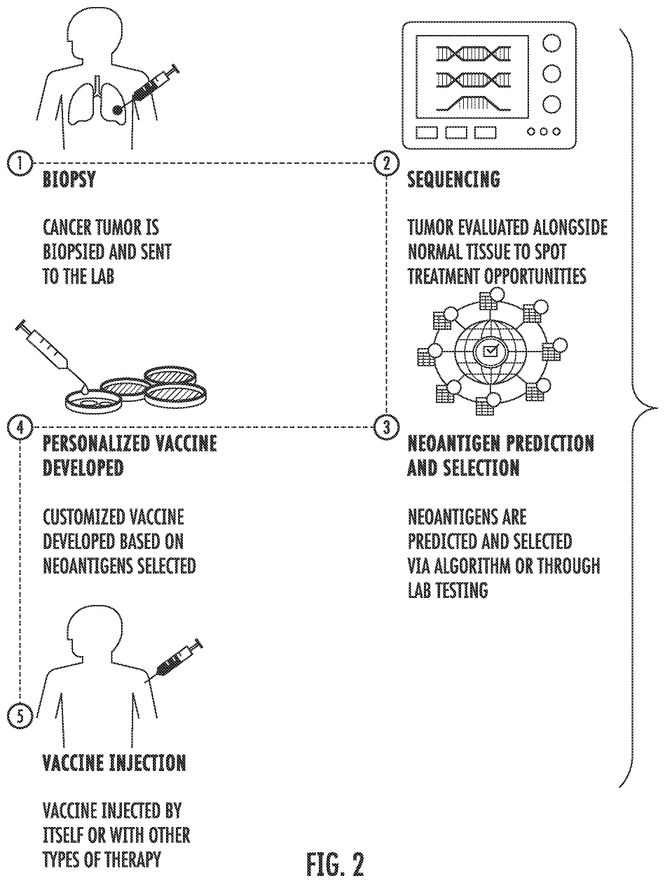

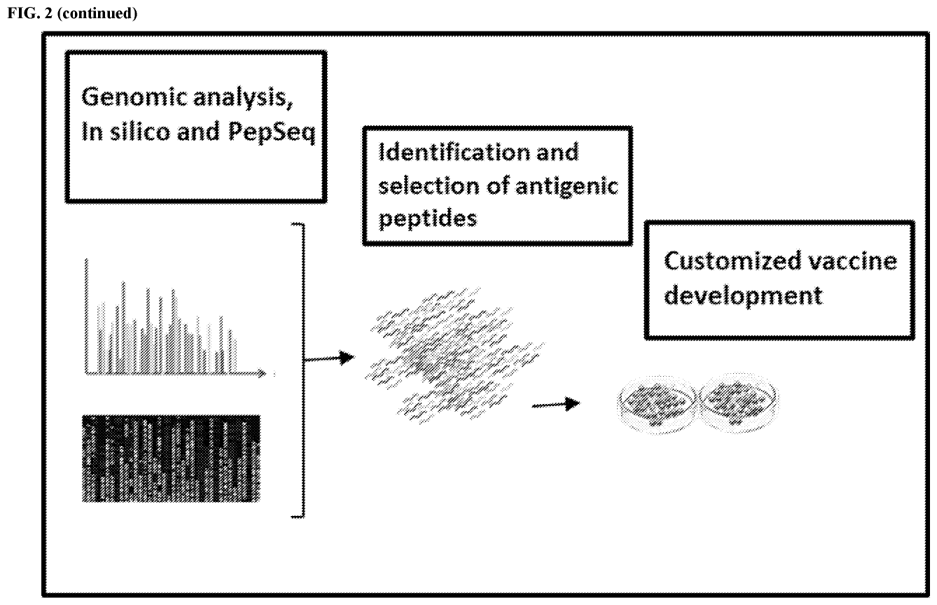

The Inventors have developed a differentiated peptide vaccine approach (FIGS. 1 and 2) in which a novel peptide:MHC screening technology (i.e., “peptide-DNA conjugate assay” or “PepSeq”) or related methodology (e.g., ribosomal display) is applied to predict personalized immunogenic neoantigens from genomic analysis. Peptides are designed corresponding to the patient's neoantigens, and then these peptides are administered to the patient. One or more oligonucleotides may be manufactured having the nucleic acid sequence that encodes a peptide corresponding to a neoantigen. The oligonucleotides may be administered to the patient.

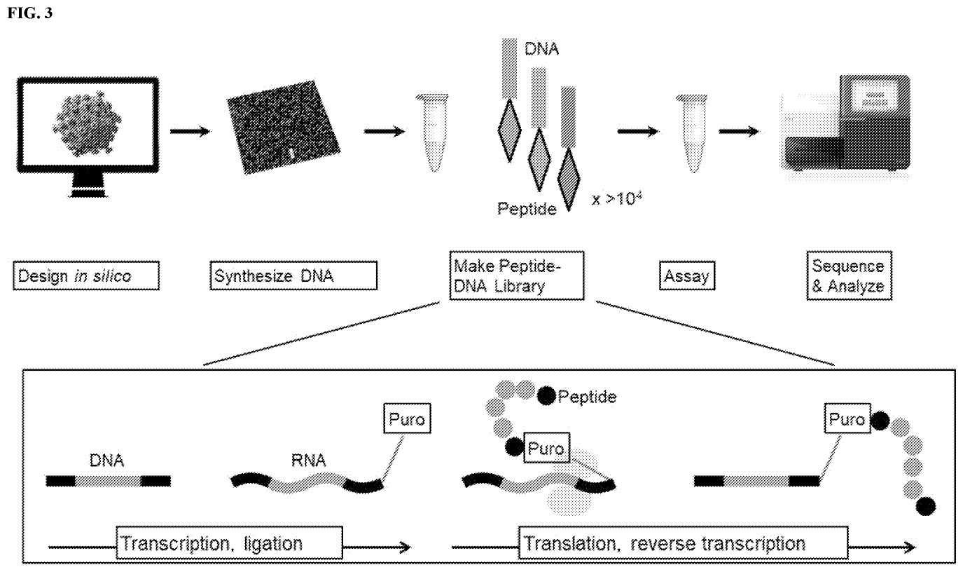

The peptide-DNA conjugate assay provides a method for efficient synthesis and analysis of large libraries of DNA-barcoded peptides (see FIG. 3).

### Example 2. A Method for the Identification of Neoantigens from Human Tumors

The following methods enable the identification of neoantigens from human tumors.

Freshly-resected tumor specimens from human patients with various malignancies will each be split into 2 samples. The first sample will be used to extract DNA and RNA, enabling whole exome sequencing and RNAseq. Together with a germline sample (e.g., blood or cheek swab), this will enable the identification of somatic variants and quantification of the expression of the associated proteins.

Following analysis of the sequencing data, the tumor genomic information will be used to design libraries of overlapping peptides covering candidate mutations and their wild-type counterparts. DNA-encoded libraries of these peptides will be synthesized in a highly-parallel fashion using the peptide-DNA conjugate platform or a phage display platform, for example, and assayed in parallel against a panel of commonly-expressed human MHC class II proteins. In parallel, in silico models of MHC class I binding will be applied to identify a second subset of candidate neoantigens. The resulting data will enable prioritization of a subset of putative class I or II-restricted neoantigens for downstream attention.

Using the candidate neoantigens identified by the peptide-DNA conjugate assay or a phage display platform and/or in silico prediction approach, two parallel approaches for enriching neoantigens will be pursued. In the first approach, selective expansion of neoantigen-reactive T cells will be accomplished by culturing TILs isolated from the tumors (without tumor cells) with peptides representing the shortlisted (i.e., prioritized) neoantigen sequences, in the presence of cytokines.

In addition, fluorescently-labeled peptide:MHC probes corresponding to the shortlisted sequences (and perhaps in combination with cellular exhaustion markers) will be used to isolate neoantigen-reactive T cells by fluorescence-activated cell sorting. In each case, in vitro activation assays will be used to measure the activity of the cellular product against autologous tumor cells, including possibly the use of in vitro-derived tumor organoids.

### Example 3. An Alternative Method for the Identification Neoantigens from Human Tumors

Freshly-resected tumor specimens from human patients are used to extract DNA and RNA, enabling whole exome sequencing and RNAseq. Together with a germline sample (blood or cheek swab), this enables the identification of somatic variants and quantification of the expression of the associated proteins.

Following analysis of the sequencing data, the tumor genomic information is used to design libraries of overlapping peptides covering candidate mutations and their wild-type counterparts. DNA-encoded libraries of these peptides are synthesized in a highly-parallel fashion using the peptide-DNA conjugate platform, and assayed in parallel against a MHC class II (DR, DP, and DQ) specific for the patient. This process enables the rapid, high-confidence identification of candidate neoantigen peptide:MHCs by selectivity and by affinity. In parallel, in silico analysis may be performed on the peptide library applying a computational algorithm to predict relative binding to MHC class I proteins based on the peptide sequences of the neoantigens. As a result, a small set of targeted neoantigens from among the typically large catalog of peptide-changing somatic tumor variants is selected.

### Example 4. Collection of Samples from Human Patients and Mice

Several human patients enrolled in a clinical trial to test the efficacy of a NeoTIL therapy. The patients had been diagnosed with lung cancer, colorectal cancer, melanoma, breast cancer, and colon cancer. In addition, a BALB/c mouse model of colon cancer was used for the collection of tumor infiltrating lymphocytes (TILs).

Tumor biopsies were collected from each human patient and from the mice. The numbers of isolated tumor cells, peripheral blood mononuclear cells, and TILs were determined for each human patient. In addition, the MHC alleles and number of mutations were identified for each human patient and for the BALB/c mouse model of colon cancer (see Table 1).

TABLE 1
Identification of MHC alleles and mutations for each human
patient and for the BALB/c mouse model of colon cancer.

| | Patient | Cancer | Biopsy | | | Number of |
|---|---|---|---|---|---|---|
| Species | ID | Type | Site | MHC Alleles | | Mutations |
| human | TG00006 | primary | lung | DRB1*07:01 | DRB1*11:03 | 83 |
| | | lung | | | | |
| human | TG00013 | metastatic | back | DRB1*03:01 | DRB1*01:02 | 374 |
| | | melanoma | | | | |
| mouse | BALB/c | colon | N/A | 1(A)d | | 1506 |
| | Mouse | cancer | | | | |
| | | model | | | | |

### Example 5. Exome Sequencing, Peptide Library Design, and Binding Assays with Human Samples

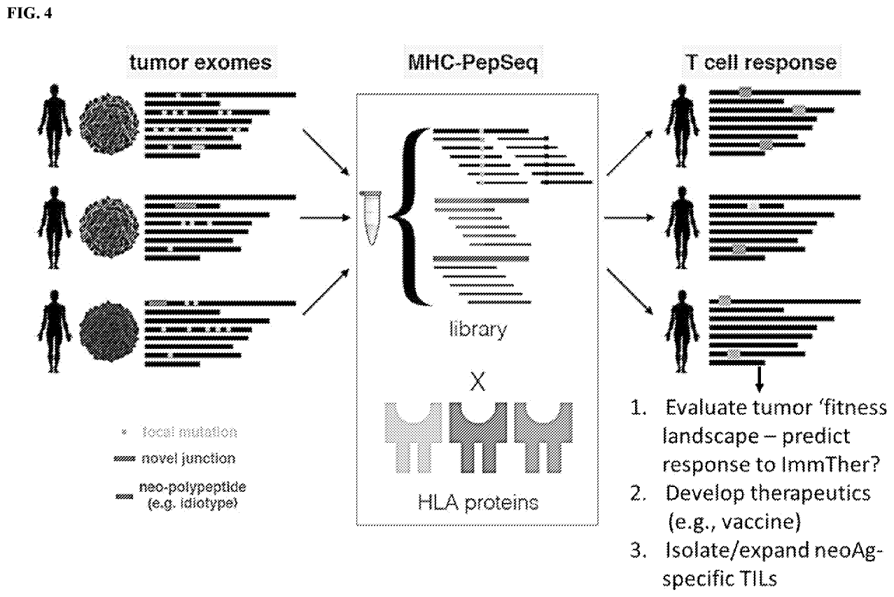

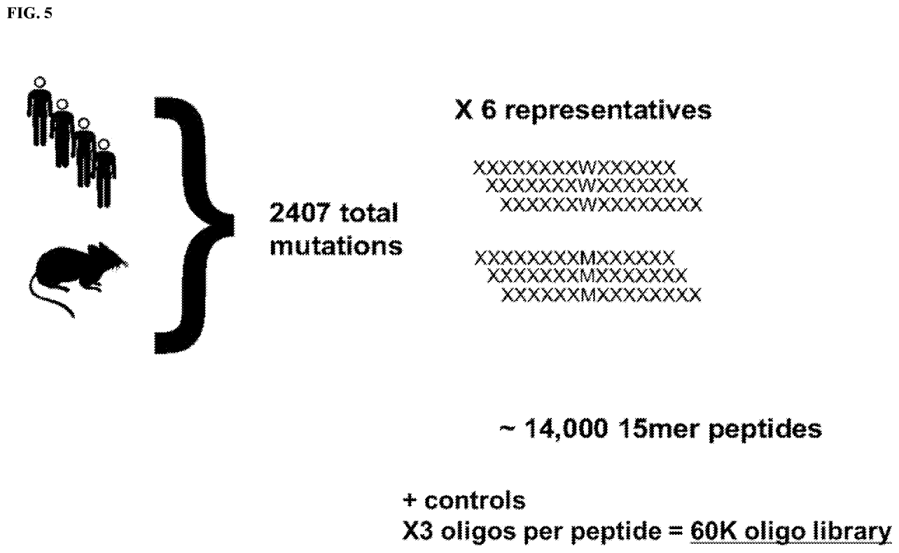

Whole exome sequencing of tumor exomes and exomes from normal cells was used to design a peptide-DNA conjugate library as outlined in FIGS. 4 and 5. Peptides in the peptide-DNA conjugate library overlapped in sequence and contained wild-type residues (“W”) and mutant residues (“M”) (see FIG. 5).

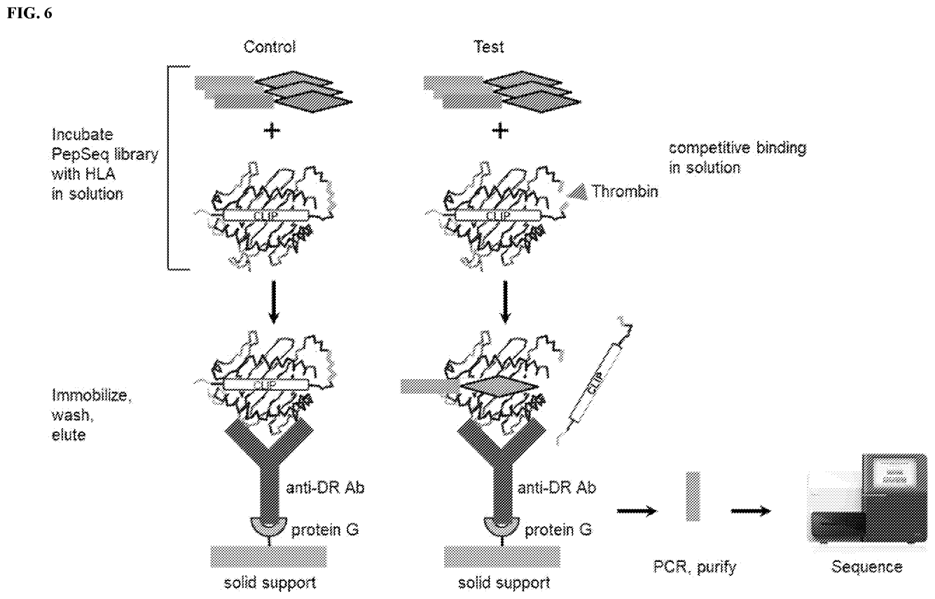

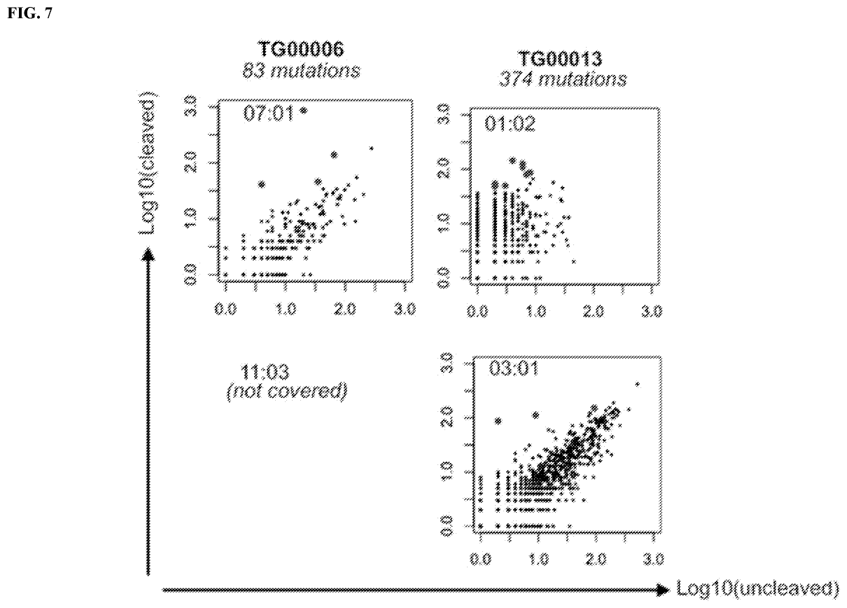

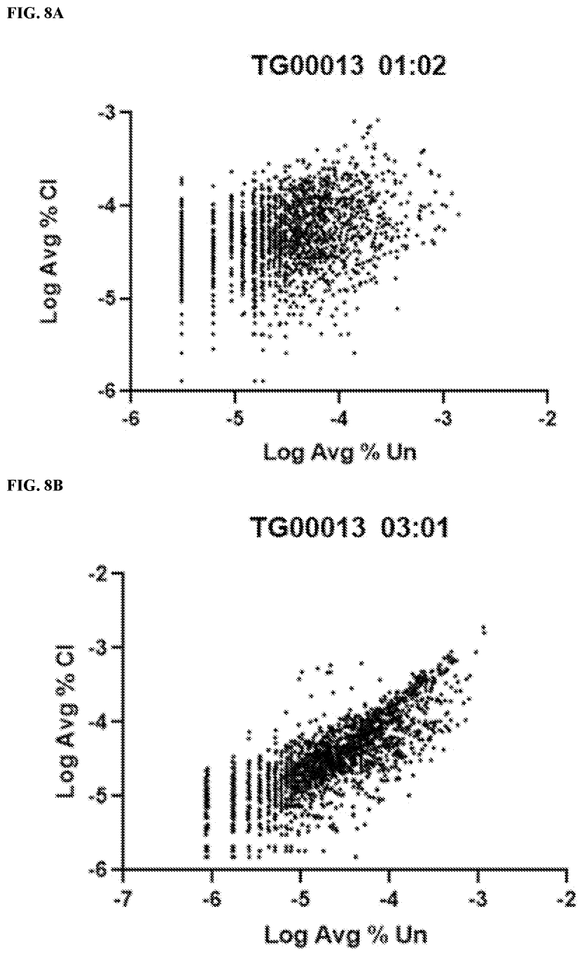

For human patients TG00006 and TG00013 a binding assay was performed as shown in FIG. 6 using the methods outlined in Day et al., J Clin Invest. 2003; 112(6):831-842. The binding assay identified peptides with high affinity for the particular human leukocyte antigen serotypes present in each patient (see the larger data points in the upper left-hand corner of each graph shown in FIG. 7). Additional analyses with the binding assay using peptide libraries for human patient TG00013 are shown in FIGS. 8A and 8B.

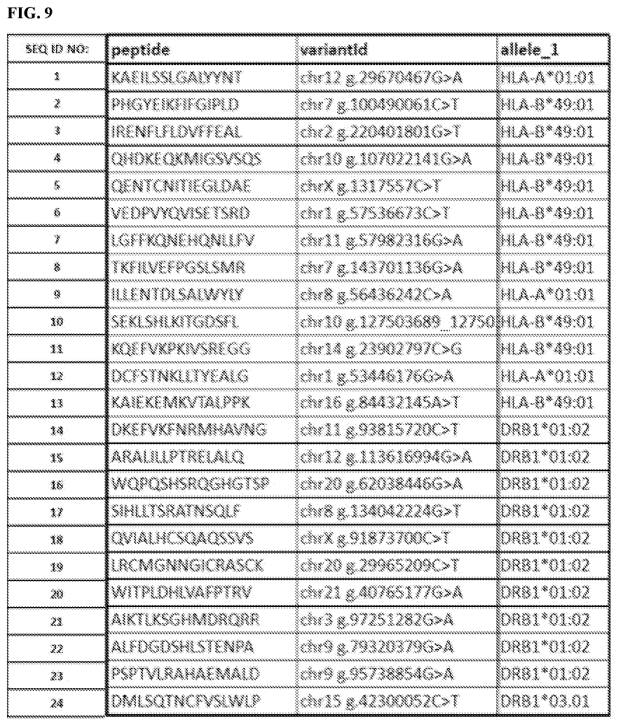

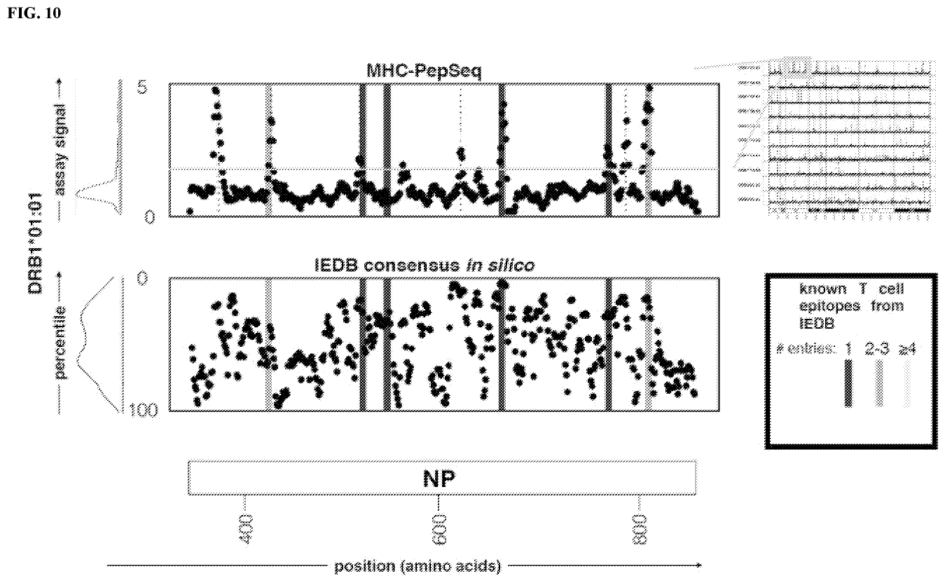

The nucleic acids encoding the peptides with high affinity for the HLA complexes were isolated with the peptide constructs identified using the peptide-DNA conjugate assay. These nucleic acids were sequenced, and the corresponding amino acid sequences are presented in FIG. 9 for human patient TG00013. Comparison of MHC-peptide-DNA conjugate assay results with in silico predictions can be used to identify and confirm neoantigens and/or neoepitopes as shown in FIG. 10.

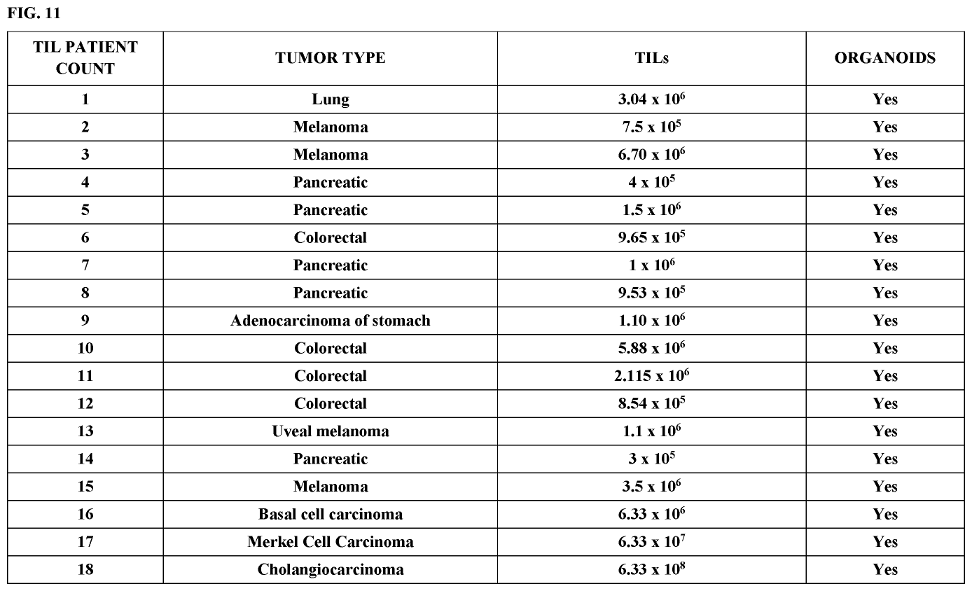

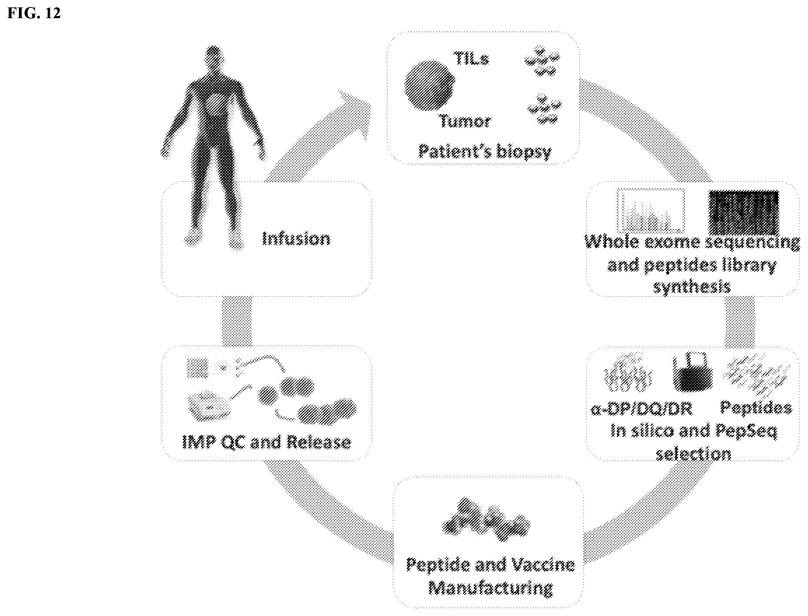

Several additional human patients with various forms of cancer including lung cancer, melanoma, pancreatic cancer, colorectal cancer, adenocarcinoma of the stomach, uveal melanoma, basal cell carcinoma Merkel cell carcinoma, and cholangiocarcinoma were evaluated by collecting tumor infiltrating lymphocytes (TILs) and generating peptides to activate and educate these TILs to enhance recognition of neoantigens and anti-tumor activity (see FIG. 11). A non-limiting example of a process for manufacturing the peptide vaccines is outlined in FIG. 12.

### Example 6. Peripheral Blood Mononuclear Cell Activation with Peptides

A peripheral blood mononuclear cell (PBMC) is any peripheral blood cell having a round nucleus. These cells consist of lymphocytes (T cells, B cells, NK cells) and monocytes, whereas erythrocytes and platelets have no nuclei, and granulocytes (neutrophils, basophils, and eosinophils) have multi-lobed nuclei. In humans, lymphocytes make up the majority of the PBMC population, followed by monocytes, and only a small percentage of dendritic cells.

PBMCs can be extracted from whole blood using FICOLL©, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes.

To test the efficiency of a peptide vaccine generated as disclosed herein, an immune infiltration assays was performed on patient-derived tumor organoids. Briefly, tumor cells isolated from the original specimen were cultured in ultra-low affinity (ULA) plate to form 3D structures (organoids). Organoids are known to recapitulate the phenotypic characteristic of the tissue of origin, including the antigenic potential. Following co-culture of PBMCs with tumor organoids derived from the same patient, the level of PBMC infiltration into the organoid is quantified by z-stacking imaging.

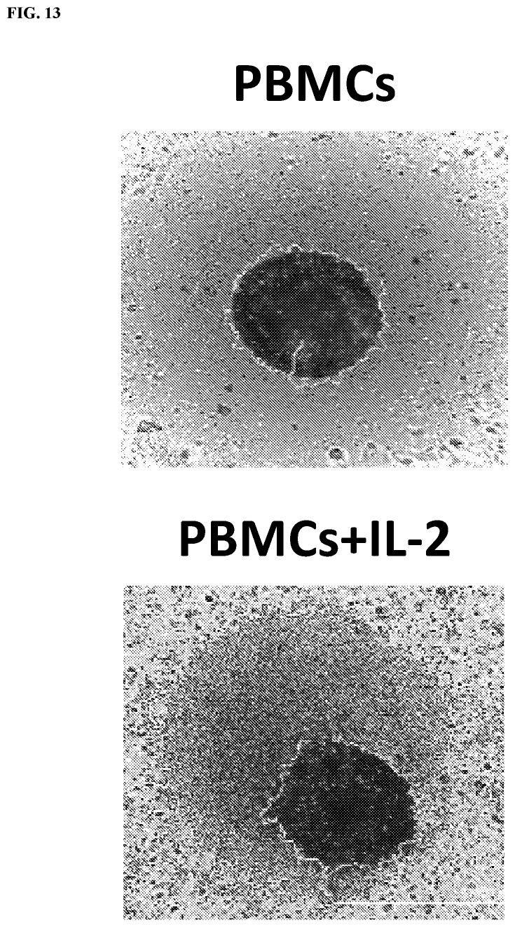

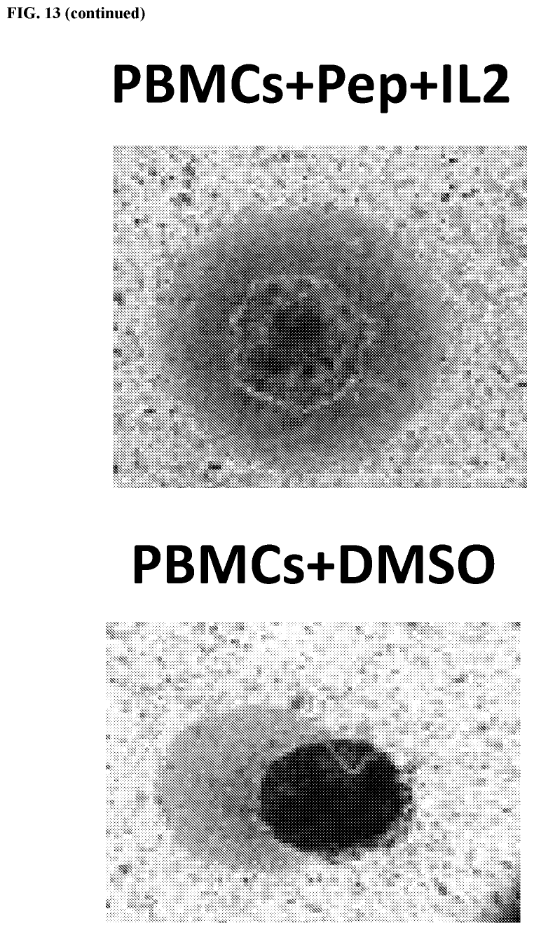

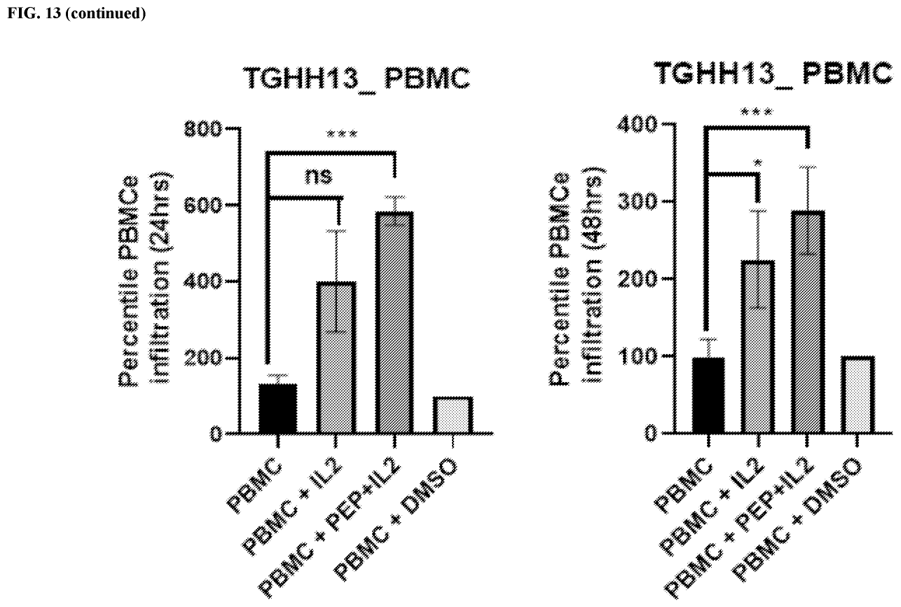

PBMCs were stained with Invitrogen CELLTRACKER™ CM-DiI, a red fluorescent dye well suited for monitoring multigenerational cell movement or location to facilitate the quantification of PBMC infiltration into the organoids. The following treatments were administered to the organoids: 1) PBMCs; 2) PBMCs+IL-2; 3) PBMCs+IL-2+peptide vaccine (“PEP”); and 4) PBMCs+DMSO (vehicle). PBMC infiltration into the organoids was quantified 24 hr, and 48 hr after administration. As shown in the bar graphs in FIG. 13, after only 24 hours the PBMCs in combination the IL-2 and the peptide demonstrated significant infiltration of the organoids.

### Example 7. Peptide Vaccine Study

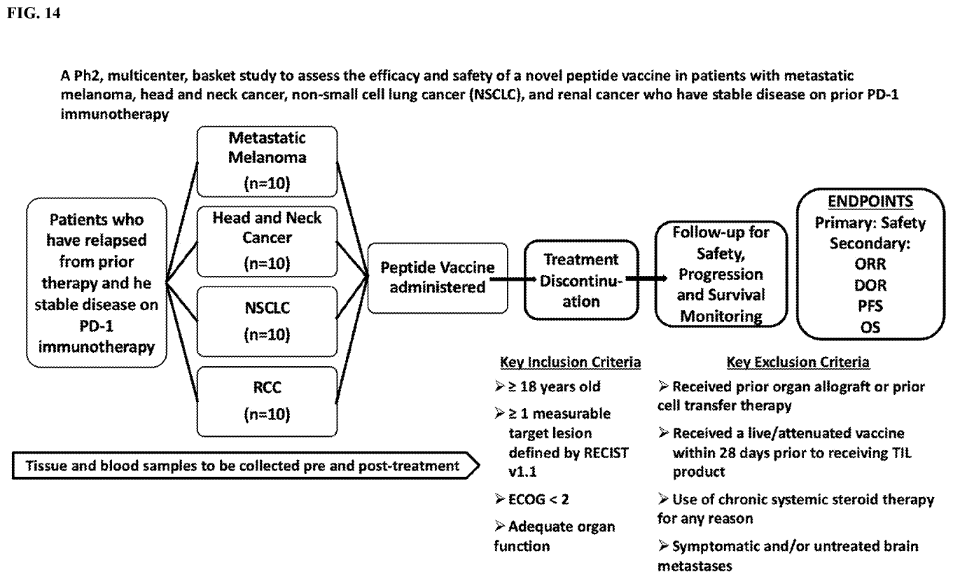

A study to evaluate the safety and efficacy of peptide vaccines produced as disclosed herein is outlined in FIG. 14. The study is conducted in patients with advanced or metastatic cancers with the key inclusion criteria and key exclusion criteria listed in FIG. 14. The study has the following objectives:
To evaluate the safety of administering the peptide vaccines;
To observe anti-tumor activity, as assessed by objective response rate (ORR) and clinical benefit rate (CBR);
To evaluate the duration of any observed response (DOR);
To evaluate progression-free survival (PFS) and overall survival (OS) for all enrolled patients;
To characterize vaccine-induced immune responses by evaluating several criteria, including antigen-specific CDS+ and CD4+ T-cell responses in peripheral blood before, during, and after vaccine treatment;
To evaluate exploratory biomarkers in peripheral blood and tumor biopsies, such as programmed death ligand 1 (PD-L1) expression, abundance and phenotypes of tumor-infiltrating lymphocytes (TILs) and myeloid cells, somatic mutational load, and neoantigen load.

The duration of treatment with the peptide vaccine will be about 12 weeks. During the Vaccination Treatment Period, safety, functional, and immunological assessments will be conducted. Safety and functional assessments will be conducted 30 days (±7 days) after the last dose.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of treating cancer in a patient, the method comprising:
obtaining tumor cells from a tumor resected from the patient;
detecting a plurality of patient-specific tumor mutations in the tumor cells with a genomic analysis of tumor DNA and/or RNA and normal DNA and/or RNA from the patient;
identifying neoantigens resulting from somatic mutations that demonstrate specific binding with human leukocyte antigen (HLA) proteins or fragments thereof corresponding to a genotype of the patient;
generating one or more peptides based on the neoantigens; and
administering to the patient a therapeutically effective amount of the one or more peptides.

2. The method of claim 1, wherein detecting the plurality of patient-specific tumor mutations comprises genomic profiling with next generation sequencing of a targeted gene panel.

3. (canceled)

4. The method of claim 1, wherein the genomic analysis comprises identifying a plurality of patient-specific tumor mutations in expressed genes by nucleic acid sequencing of tumor and normal samples from the patient and the mutations are present in the genome of cancer cells of the patient but not in normal cells from the subject.

5. The method of claim 1, wherein
the plurality of patient-specific tumor mutations comprises a point mutation, splice-site mutation, frameshift mutation, read-through mutation, gene-fusion mutation, insertion, deletion, or a combination thereof, and
the plurality of patient-specific tumor mutations encodes at least one mutant polypeptide having a tumor-specific neoepitope which binds to an HLA protein or fragment thereof with a greater affinity than a wild-type polypeptide; and, optionally,
further comprising identifying Major Histocompatibility Complex (MHC) class 1 and 2 genotypes of the patient.

6. (canceled)

7. (canceled)

8. The method of claim 1, wherein identifying the neoantigens comprises:
(i) providing a library of peptide constructs, wherein
each peptide construct of the library comprises a peptide portion and an identifying nucleic acid portion that identifies the peptide portion, and
the peptide portion of at least one of the peptide constructs is capable of specific binding to the HLA proteins or fragments thereof,
(ii) contacting the HLA proteins or fragments thereof with the library of peptide constructs;
(iii) separating the at least one peptide construct comprising a peptide portion capable of specific binding to the HLA proteins or fragments thereof from peptide constructs comprising a peptide portion not capable of specific binding to the HLA proteins or fragments thereof, and
(iv) sequencing all or a portion of the identifying nucleic acid portion of the at least one peptide construct capable of specific binding to the HLA proteins or fragments thereof.

9. (canceled)

10. (canceled)

11. The method of claim 1, wherein identifying the neoantigens comprises:
(i) generating a genetically encoded combinatorial library of polypeptides with phage display, ribosomal display, mRNA display, biscistronic DNA display, P2A DNA display, CIS display, yeast display, or bacterial display, wherein the combinatorial library comprises polypeptides linked to corresponding nucleic acid molecules encoding the polypeptides;
(ii) contacting the combinatorial library with the HLA proteins or fragments thereof;
(iii) separating HLA proteins or fragments thereof demonstrating specific binding with the combinatorial library; and
(iv) sequencing all or a portion of the nucleic acid molecules of the combinatorial library bound to the HLA proteins or fragments thereof to identify the neoantigens; and optionally,
wherein the combinatorial library of polypeptides comprises variant peptides designed from an analysis of the plurality of patient-specific tumor mutations predicting the impact of each mutation on a corresponding protein and excluding silent mutations and mutations in noncoding region.

12-15. (canceled)

16. The method of claim 1, wherein specific binding between neoantigens and HLA proteins or fragments thereof is determined by:
(i) culturing a cell transformed with at least one nucleic molecule comprising a nucleotide sequence encoding:
an MHC class II component comprising at least a portion of an MHC class II a chain and at least a portion of an MHC class II 3 chain, such that the MHC class II a chain and MHC class II 3 chain form a peptide binding groove; and
a spaceholder molecule and a first processable linker, wherein the spaceholder molecule is linked to the MHC class II component by the processable linker and the spaceholder molecule binds within the peptide binding groove thereby hindering the binding of any other peptide within the peptide binding groove; the step of culturing being conducted to produce the MHC class II component;
(ii) recovering the MHC class II component;
(iii) processing the processable linker, thereby releasing the spaceholder molecule from the peptide binding groove;
(iv) incubating the MHC class II component in the presence of a neoantigen, wherein the incubation facilitates the binding of the neoantigen to the peptide binding groove;
(v) recovering the MHC class II component that has bound the neoantigen; and, optionally,
wherein the spaceholder molecule has the consensus sequence AAXAAAAAAAXAA (SEQ ID NO: 30).

17-22. (canceled)

23. A method of treating a patient having a disease, the method comprising the step of:
obtaining tumor cells from a tumor resected from the patient;
detecting a plurality of patient-specific tumor mutations in the tumor cells with a genomic analysis of tumor DNA and/or RNA and normal DNA and/or RNA from the patient;
identifying a neoantigen resulting from somatic mutations that demonstrate specific binding with human leukocyte antigen (HLA) proteins or fragments thereof corresponding to a genotype of the patient;
designing a peptide based on the neoantigen;
generating a nucleic acid sequence that encodes the peptide; and
administering to the patient a therapeutically effective amount of an oligonucleotide having the nucleic acid sequence that encodes the peptide.

24. The method of claim 23, wherein detecting the plurality of patient-specific tumor mutations comprises genomic profiling with next generation sequencing of a targeted gene panel.

25. The method of claim 24, wherein the genomic profiling comprises whole genome profiling, whole exome profiling, and/or transcriptome profiling.

26. The method of claim 23, wherein the genomic analysis comprises identifying a plurality of patient-specific tumor mutations in expressed genes by nucleic acid sequencing of tumor and normal samples from the patient and the mutations are present in the genome of cancer cells of the patient but not in normal cells from the subject.

27. The method of claim 23, wherein
the plurality of patient-specific tumor mutations comprises a point mutation, splice-site mutation, frameshift mutation, read-through mutation, gene-fusion mutation, insertion, deletion, or a combination thereof, and
the plurality of patient-specific tumor mutations encodes at least one mutant polypeptide having a tumor-specific neoepitope which binds to an HLA protein or fragment thereof with a greater affinity than a wild-type polypeptide; and optionally,
further comprising identifying Major Histocompatibility Complex (MHC) class 1 and 2 genotypes of the patient.

28. (canceled)

29. (canceled)

30. The method claim 23, wherein identifying the neoantigen comprises:
(i) providing a library of peptide constructs, wherein
each peptide construct of the library comprises a peptide portion and an identifying nucleic acid portion that identifies the peptide portion, and
the peptide portion of at least one of the peptide constructs is capable of specific binding to the HLA proteins or fragments thereof;
(ii) contacting the HLA proteins or fragments thereof with the library of peptide constructs;
(iii) separating the at least one peptide construct comprising a peptide portion capable of specific binding to the HLA proteins or fragments thereof from peptide constructs comprising a peptide portion not capable of specific binding to the HLA proteins or fragments thereof; and
(iv) sequencing all or a portion of the identifying nucleic acid portion of the at least one peptide construct capable of specific binding to the HLA proteins or fragments thereof; and, optionally
wherein the library of peptide constructs comprises variant peptides designed from an analysis of the plurality of patient-specific tumor mutations predicting the impact of each mutation on a corresponding protein and excluding silent mutations and mutations in noncoding regions.

31. (canceled)

32. (canceled)

33. A method of treating cancer in a patient, the method comprising:
obtaining tumor cells from a tumor resected from the patient;
detecting a plurality of patient-specific tumor mutations in the tumor cells with a genomic analysis of tumor DNA and/or RNA and normal DNA and/or RNA from the patient;
identifying neoantigens resulting from somatic mutations that demonstrate specific binding with human leukocyte antigen (HLA) proteins or fragments thereof corresponding to a genotype of the patient;
generating a messenger ribonucleic acid (mRNA) polynucleotide comprising an open reading frame encoding one or more peptides based on the neoantigens; and
administering to the patient a therapeutically effective amount of the mRNA polynucleotide.

34. The method of claim 33, wherein the mRNA polynucleotide comprises at least one chemical modification.

35. The method of claim 34, wherein the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-i-methyl-pseudouridine, 4-thio-pseudouridine, 5-aza-uridine, dihydropseudouridine, 5-methoxyuridine and 2′-O-methyl uridine.

36-40. (canceled)

41. The method of claim 33, wherein detecting the plurality of patient-specific tumor mutations comprises genomic profiling with next generation sequencing of a targeted gene panel.

42. (canceled)

43. The method of claim 33, wherein the genomic analysis comprises identifying a plurality of patient-specific tumor mutations in expressed genes by nucleic acid sequencing of tumor and normal samples from the patient and the mutations are present in the genome of cancer cells of the patient but not in normal cells from the subject.

44-46. (canceled)

47. The method of claim 33, wherein identifying the neoantigens comprises:
(i) providing a library of peptide constructs, wherein
each peptide construct of the library comprises a peptide portion and an identifying nucleic acid portion that identifies the peptide portion, and
the peptide portion of at least one of the peptide constructs is capable of specific binding to the HLA proteins or fragments thereof;
(ii) contacting the HLA proteins or fragments thereof with the library of peptide constructs;
(iii) separating the at least one peptide construct comprising a peptide portion capable of specific binding to the HLA proteins or fragments thereof from peptide constructs comprising a peptide portion not capable of specific binding to the HLA proteins or fragments thereof; and
(iv) sequencing all or a portion of the identifying nucleic acid portion of the at least one peptide construct capable of specific binding to the HLA proteins or fragments thereof; and optionally
wherein the library of peptide constructs comprises variant peptides designed from an analysis of the plurality of patient-specific tumor mutations predicting the impact of each mutation on a corresponding protein and excluding silent mutations and mutations in noncoding regions.

48. (canceled)

49. (canceled)

50. The method of any one of claims claim 33, wherein identifying the neoantigens comprises:
(i) generating a genetically encoded combinatorial library of polypeptides with phage display, ribosomal display, mRNA display, biscistronic DNA display, P2A DNA display, CIS display, yeast display, or bacterial display, wherein the combinatorial library comprises polypeptides linked to corresponding nucleic acid molecules encoding the polypeptides;
(ii) contacting the combinatorial library with the HLA proteins or fragments thereof;
(iii) separating HLA proteins or fragments thereof demonstrating specific binding with the combinatorial library; and
(iv) sequencing all or a portion of the nucleic acid molecules of the combinatorial library bound to the HLA proteins or fragments thereof to identify the neoantigens; and, optionally,
wherein the combinatorial library of polypeptides comprises variant peptides designed from an analysis of the plurality of patient-specific tumor mutations predicting the impact of each mutation on a corresponding protein and excluding silent mutations and mutations in noncoding regions.

51-57. (canceled)


SEQUENCE LISTING (30 sequences)

SEQ ID NO: 1 - protein, 15 aa, Artificial Sequence
  Neoantigen
KAEILSSLGALYYNT

SEQ ID NO: 2 - protein, 15 aa, Artificial Sequence
  Neoantigen
PHGYEIKFIFGIPLD

SEQ ID NO: 3 - protein, 15 aa, Artificial Sequence
  Neoantigen
IRENFLFLDVFFEAL

SEQ ID NO: 4 - protein, 15 aa, Artificial Sequence
  Neoantigen
QHDKEQKMIGSVSQS

SEQ ID NO: 5 - protein, 15 aa, Artificial Sequence
  Neoantigen
QENTCNITIEGLDAE

SEQ ID NO: 6 - protein, 15 aa, Artificial Sequence
  Neoantigen
VEDPVYQVISETSRD

SEQ ID NO: 7 - protein, 15 aa, Artificial Sequence
  Neoantigen
LGFFKQNEHQNLLFV

SEQ ID NO: 8 - protein, 15 aa, Artificial Sequence
  Neoantigen
TKFILVEFPGSLSMR

SEQ ID NO: 9 - protein, 15 aa, Artificial Sequence
  Neoantigen
ILLENTDLSALWYLY

SEQ ID NO: 10 - protein, 15 aa, Artificial Sequence
  Neoantigen
SEKLSHLKITGDSFL

SEQ ID NO: 11 - protein, 15 aa, Artificial Sequence
  Neoantigen
KQEFVKPKIVSREGG

SEQ ID NO: 12 - protein, 15 aa, Artificial Sequence
  Neoantigen
DCFSTNKLLTYEALG

SEQ ID NO: 13 - protein, 15 aa, Artificial Sequence
  Neoantigen
KAIEKEMKVTALPPK

SEQ ID NO: 14 - protein, 15 aa, Artificial Sequence
  Neoantigen
DKEFVKFNRMHAVNG

SEQ ID NO: 15 - protein, 15 aa, Artificial Sequence
  Neoantigen
ARALILLPTRELALQ

SEQ ID NO: 16 - protein, 15 aa, Artificial Sequence
  Neoantigen
WQPQSHSRQGHGTSP

SEQ ID NO: 17 - protein, 15 aa, Artificial Sequence
  Neoantigen
SIHLLTSRATNSQLF

SEQ ID NO: 18 - protein, 15 aa, Artificial Sequence
  Neoantigen
QVIALHCSQAQSSVS

SEQ ID NO: 19 - protein, 15 aa, Artificial Sequence
  Neoantigen
LRCMGNNGICRASCK

SEQ ID NO: 20 - protein, 15 aa, Artificial Sequence
  Neoantigen
WITPLDHLVAFPTRV

SEQ ID NO: 21 - protein, 15 aa, Artificial Sequence
  Neoantigen
AIKTLKSGHMDRQRR

SEQ ID NO: 22 - protein, 15 aa, Artificial Sequence
  Neoantigen
ALFDGDSHLSTENPA

SEQ ID NO: 23 - protein, 15 aa, Artificial Sequence
  Neoantigen
PSPTVLRAHAEMALD

SEQ ID NO: 24 - protein, 15 aa, Artificial Sequence
  Neoantigen
DMLSQTNCFVSLWLP

SEQ ID NO: 25 - protein, 15 aa, Artificial Sequence
  Spaceholder
PVSKMRMATPLLMQA

SEQ ID NO: 26 - protein, 13 aa, Artificial Sequence
  Spaceholder
AAMAAAAAAAMAA

SEQ ID NO: 27 - protein, 13 aa, Artificial Sequence
  Spaceholder
AAMAAAAAAAAAA

SEQ ID NO: 28 - protein, 13 aa, Artificial Sequence
  Spaceholder
AAFAAAAAAAAAA

SEQ ID NO: 29 - protein, 13 aa, Artificial Sequence
  Spaceholder
ASMSAASAASMAA

SEQ ID NO: 30 - protein, 13 aa, Artificial Sequence
  Spaceholder
  misc_feature (3)..(3): Xaa can be any naturally occurring amino acid
  misc_feature (11)..(11): Xaa can be any naturally occurring amino acid
AAXAAAAAAAXAA